SEQUENCE LISTING (46 sequences)

SEQ ID NO: 1 - DNA, 1020 bp, synthetic construct
  source 1..1020: mol_type = other DNA; note = gene gK
tcaaggcccgaacaatttgttttgtatcttctgttcgtatttaaatgcaattattacaatgctcgcaatcgcagccacgcacaatgcacgcaagactaagctcgaagcaatattggcaaggcatgaagtcaagatactgggaggtttcgatgcggagactttagtctttgccggagtcgtgtaagggatcagtgataagagttctagaaccgctgagacaattacatatacccaagcactaatttttacgtgttcatgtaaaatatatgcacacggatcatagcaaatccgcaatgtcatggtggacataataagcgatgctaaatataaaccgatctcgagtgttttcagtagtaagacccctggattacagacgcaaaatcctagagggtctgctacgtagttcaagcttttgcaatctcggagatgttcaaattccctcaaaaaccgttgcatctttgtgtatggcacccgtaatatcagcctcgaagtaaacgctgtccagtagttaagatgatatgcatccatcgcaatgcgttctcgcggcagacatagcccgaacattcgcctctggcgacgggtcagcaagtaacatatgtacaataccgtcgaaatgaatgcaactctgatctgggaaaaccatatgtaaagcagacagtctgccacttccatgacgaatactgttctttggccggtgggatcaagacgagaaatgagtacagtctcattttcaaaataagctcctttaacacatctcgtctggtggttctcgtagttgtttagacgctgtgtatattttgtgaggacataaactagtgaagaattaaatgtagcggggggttggtgaacaggattgaattcttgctgtccgggcaggagccatttaatatttgcgagtacgtacaagcagtcgtcctttgagtcagtggaaattttagagcatacataccataccaagaagactatatgtatcgttccggttaaaattactgcccattttagacattctggtcgcagcat

SEQ ID NO: 2 - DNA, 879 bp, synthetic construct
  source 1..879: mol_type = other DNA; note = gene gG
ctactgctggagcgtagagggcggtgaaaagacaaccatcctgggcatcgtctctgacctttcgtcgacgtactcgtccagcggaccggcgcattcctcgtcccataggtaaagctcgtccgtggcgttttcctgctcctcgacgtcatgcgaacaaacggttgtccatgggcgtagcgggggcatgtctagacaattggaataatcttcctccataggaccgtcgagttcgggcgaggctacgtcataagatgataagtctgacgcctcgtcggccaggctgtaataggtgtacccatactggtcgtggcatccggggtttggtccgacgtctagataaacagacccggtatatcccttcccacccactgagagctggtaggcgtagatgccggcatcgtacatccctgggtacagcaccagggatccattgccagacacgtaactaccaagttccaccccggagcgcagtcttctctgcactttttcgcactgcttgtcgaaatcaggtatcccggcgtcagacgccctrcacacagacgcgttcagattatggattagagcccagcacgtagacaggtcctcgtgtccactgcccgtttcgctatccaccccgaacacgaagctttcgggattactattcccgtagaaccatttcacctcgactgacactccttcggtgcgaccagagtagtcgctccacgggaccgtcacctcaatcgtggtgcggttgcacgcctgggctataattttgcccccgaacgggaaagggctcgactcgagcccgtccagtaccggcgcccctaatacagatgcgcacaaaagcgagcatactagggaaacggtggcaatcgatcctatgttactgaagccgctcat

SEQ ID NO: 3 - DNA, 1500 bp, synthetic construct
  source 1..1500: mol_type = other DNA; note = gene gE
ctaatagctatccatattccttctgccgcttttatttgccaggatgcgatctgccattgatgcgatggcggatttaaagtttaaggccctttctccctccacgaccgcaccgaaaaaggtattcacatcttcgcggaattcctgcttacagtagccgcgaaataggggttcgtatacttcgtcataaatcacttcctcatcgtccgtaagcccgccggcctcaaactcgtttttggggagcggcgcgtacaatggttcggtcttggaaaaagtacccatttggtgcgcggggcacttctttttccccacaagcagagcgcatgtaagaattacgcacgtcgcggtcacccccgcgacgacgaagtacattttatagttacccgtttcgtccattggtccgggactgccaattactactttgttcatcgggaccttcgagggcatgtgcgtttcgactactacgcgcggactgttacgtttcccgagcaccgtgtagctccatcccaaaatgtctccgtcggaaacgactataatcaaaactttggggatggctataagaggactgatcaccaggcctgcgcgtgactcggagagtcgcgaatcgaaccgtaggttcggcacgcgattttcgtggcagtcttctagagtatcgcgggaacaatccagcaaagaggccgcgcccaagaattccgccatcaagacagatcctatgaggcatctctggttggttgcgggaaactgacatttgccgtctgtaggatggtatacgcacggctcgaacagtttcacttcttcgcactcctctttgacatcaaacccataccacgaatacgtaacgttaaattttacgtgctcggtcggtgggaagactctaagaacaggggtcacataggtggggctgttgtcgttgagttcatgaaacggcagcatttgtaattccacgtgcgcgtgggcctctgacggcttgatggtgtctatctccacctcgtgcggtgctggagccttctcgtctacgtgcgaaagaacgctgacgttgtacgatgtagtttccatggccagatctggctgagagatccgtcgaaaatagattccctcgtccgaacgagttacaggatcaaagagcaggcctccgtactctgtccacatgatattagttatattgctgttgtgtagcccagaacccagcacgacgaagctttcgccgcgctttagtaaaattaccaggtttgcagacaggggtcccgcgggcgggcagtctttctttcctccgtcgtaaattaaatcatgaaagcaatagttaggcgaagagcagacatcggccgccttcatcttgcacgcgttccgcaggacttttgtccatttgattacagtcccttccggaacgtttctgggatcatcttccccgaggacagcctgagtgccttccaaaaagaggacgtgtcgcgtcgcaaaagttaggcgcgcaagacaagaggcgagaactatcactaacatgttcat

SEQ ID NO: 4 - DNA, 1749 bp, synthetic construct
  source 1..1749: mol_type = other DNA; note = gene UL32
atgacatccaaacgatcaactgcagaaactcgcactgaactttccaagggatggaaagctggagtgctctcaaaactatatactggctacgacccggcgattctgacgtttaatgacaagttgtcggacgaactattgtatggagctcacctaattgaaatatatcatgaagaaagtacagaacaagctgaagaacaaaacagaattccggacattgattctttcgtagaaaccatggctgacttatacaccctagataaatcatgcgccgtctgcaagattattaccaaatatcaaaaaaaattccccgtgactcccgaatggatggtggactactctttattgtgcttcaaatctcaaagcatccccctgtgtgctacatctaccttcataactgcttttgagttcatcttcatcatggacaaacactacttgcattcccacagcacctctttagttggcgctataagccgtcgggaattttccctcggggatattcagaagcacttcttttttaacggctgtttcaaagcggtggaagggggactggactctaaaatagatctaaataattattcgttcctagtgcagtcggtagcacgctatgcgttactatctactagatatcaagaagcacttagggcaaaaattgccccggtctctggcgcgtctgcagatttcaacttccaaaaaagtagcggaatggccgtggctttgttaaaatggcgtgagtatgccagaccactcgagtgtttttgctccgcgcagtgtatgttaaagaggaaaaatacagagactgcctccttcgcttttcgaacctgtgcgcaaaaagctgctttagaagatattagcacggagtcaaaatatgtggaagggggaccaagcgaaaccctgacaaccccaagtaaatggggatttaccgacctgacggcattattaattgctggaacagccggtatgccaaatcgggaactcgatgccagctgcagcgacgagcctgagtatgatgatacaaatgataatggagaaaatttcgttgcccctataattcggacaagaaataccgtagtggagaactttcacgcaacgctaatccaacaggtcaaaaatactaggctccccaagacagcgaagcagtacatttgcgacaataccaaaccatcacttgccattggcccaattttggcgtctattttgcctcacccgactagccgaggagatactggaggggaatgcgtgttgtgcaatctcatgcttactcgggaacactggcatgctcttcgaaagctgaaaagtaaagtagtcggctgcagtaacgtaaatagcagcttgttcgatgggattgaaccagcattagaaacgttcgaagactatactgcgttaaacgatggggggcgaatgttaaccttattgaggatggccggggcaaacgcaatttacaaacactttttttgcgatccattgtgtgcagttaatacgcttcgcgtaaatccaaaagttttgtgggaaggccaacccaaggacccagaactgctgcagctttataaagccgaaatcgccacggctaacatatttcaagaacgggtttgccgcgggctgtggattctagccttcacatttaaagcttaccagctttctcctcctcgccctaccgctttaaattctttcattcgcggcgctgagacctatttaaagaggcacggaatcagctgcattgctttagaacacgcactgactcgatatgtctga

SEQ ID NO: 5 - DNA, 4450 bp, synthetic construct
  source 1..4450: mol_type = other DNA; note = gene ICP4
atgtcctctgattcctcgacgccgagtaatttggccgctccaattgttgatgagtcgggattctcaactggcccgccctcagatctatttacctttttgcgcgccgataagagtgggcgctacgattctttgggttctcccatcccgaaccctccaaacgcacctacctgtgagaccactactgatagcttttcgtacagcacgccacctcttttttccagcgaggacgagctctttcactctgtcatcgctgagaatgcggcccaagacgtggacgggcgagaaatgtctctccgcgaccttatagcgatgttaaacgacatggatcccgaaaacataaactctatagaaccgtgtccgaacgctaccagtgatgtcgtggcactagatattaacgtggataccgatcagcccggagtttatacagtcatcgactcgctcgcacatccctgcgaaactcccatggagcagcagcacgttgaatgcccgtgtccattacctgcagtagagaaaacaaccgctgtgaggcgtcaagaagtggtgaggaagtcagagagagtggccagatctcgttcgcgacgcgccatggcgcgatctttgagcaaccggcgccccgtcccttcgccgcagaggaccagcaaagaccgtctccccaagcgagggaagagggaattttcgaagaagatgggcccgagccaccttacctcttcctcctcttcctcctcttcctcattctcgctgtcagggaggaggggacgtctcgcccgaaggctggaaagcgcgggaagtgaaaaatccgccgaaagccccgaccgggactctgcccaattagtgctacctggagaatgtcccgatgtcgcgctagagccactctggcgagttttgagggagtgggtcgaaacggacggtacagacgggtcggttcagtcagtaaccgcgcctacggagctcccagacctaggccgcggcattaccctgaatgtactctcacgagcgttggccgtcgacctccatagttccgaggctcgcgtttttgttccagaagctattatcccgtccccacccagtagaggacaagtatatcatggaagtggctatagacagtctttggtcgatgacccgcaggttcgggaggccgcggccgaattccatgccaatccgaaatcagcttccgtgtatctcgaagagtacgggatgccagtggaacagttagagcgccttgtggaggagttcatggtccagcccctgtcgcgcgtttcgtatgggtatggcggccccaagctgagcccacgtgacaaacggctctgcgaactgtgcgctctccgcggtccgggttcttccgagggaggattgtctcgttctgcggtgccgacgtgcctcccgcacgtagcgcacgccatgcgtgatggccgtgtgtcattagctctgccgcaccttaccgagctcgtacgcgtgtgccgccgctatgaccatccgcagaaaacttacctgttggcggctcttaggagagcgtttgtcccgtttgtatttccaggagcggccgtggatcctacggcggcgtctgcttctggggaccaccctgtactcgttggcgccaggcgaatagcgcgtgccacgagggagctggaagccgcagcgaaagagccacctgcggcaaccccggaaaaactttcctcttcggcgctattgcgccatactagaacttgcattgttgctgccgcagatctcatggcggcgcttactgctatagactgcccaccagttacaataaaccccgccaagcgtcgcccggatctgtatcaagcactctcgagggcgcccaaaacagtctttacgtgggagcgcgccatgcgctactgcgcggcttcgctatggagtcgcctcgtcatagggtccctgccgctgccatctaccgatgatgtcactcttcttgtagaagccatggaagctgccgtgggcgtgcttggctgctccgacgagggtgacgtgcggccttcaattgcctacgcggcctctggttggaggatgatcgctaccatgctaggagatgattccgcgcccggcagcgagcacgtcacggacgattgtcccgcagatgatgacacccaccaccatgacaggcgcaaaagacgaggcggccggcggagcacttgccggtacgacctacctcctggcactcccgatcgcgcggcccacctcctactctacgggagtcctggcggcggcaacggagcagagccttcggctgcccaagaatctcccgccaacccctggcccagagctccaccgtgcgatgaacaggagccattatctgtgtcaccgtacgggcccgaacccgaccgccctccagatgatgactttgagacccgtaaaggtctgaaaagaaagtcatctgaagatcacgcggacccgatcccagaaggcaacgcgactaaaaaaacatgtggcctccagggcttaccagattcattacctccagcggttccagaaacagatagagacaacctcctcctttcgccatgccccataactcccgagggtccaccatgtccgccccgagaagaaccgcaagaaccacaatccccgtcctttcacatttccgagataggtgaggccctgtttcattctaccccaatcagccctaccatcctgttcgcaccggagggtttcatcccgaaccaaggggatgtttaccgtccccatggcaaaaacttgggcgttgcgctctccgcaggggagccatacgtgcccgcccatcgcgcgcgtcctgctttggagcggctcgcgaactatttgcgcgggttgagcagcggtccggcgggaagctattccgcgaccggacccgcagattggctacctgcttttgcgagggacccagtagccctgggggagttttgcaagaggatttcacccgtgaacagaggggggcgctgcgtcttagaggagcggctttcctggacgttgcgacatccggctgacaaaatctatgacctcatcattcttgtctcagaacgtgccccgggcgaatttttagaacgcgcgtacgcctacgcggccctctgcggacgtgcctgcatgccgggcggtgcctgttggccagaatcgtggacacgcggggagtatccctatatccctcccgacgagaaagacccgggccgcagcgtggccattctctgtacgacagaccttggatacgccggggcggtagaatggatgcttgagcgcgctctcagtacatctgggcgcgcatgcgtggtcgtagatgctagggatcccggggacgaggattcccgctcgggtccgcgcatacctaccgggcggaggggtgttgtctatgcccggactagtcccccgtcgcacgcttaccttgtaaaactgtttaccggtccggtggccgaacggttggaacggggtgggctcaatgtaggtcacgtagtaatggacaggcgactcactgtctcatggcccctcagcgttgggaagaccgacaacgacgatgagggtacaggagacagagagaagtgtcctcccgttaagcaccccttcctcactcacgtaactctcaggagactacgcgacgttgctcgtgccatggatgccgggtggagccactccgaaatcggaaaagcttcagccgtcttgtatccgtacatggcccagccccatgtgggcgccggggcgatgccgccatgtccggacctttccgagtccagttccactatgcattcttcttcgtcgtcttcctcttcctcatgctcctcgtcgtcgtcttcttcagactccagctcatctgaagaagacggggacgagaagaacgagaaagaagatcgtgagcgcgccggaggtggaaagcggcgcggccgccagcgcctcctgattcgggaccgcgtgtaccgtggccgggactaccgcgtgcgcactccctgcgctgatattgtgggaggcacttccgcttcctttagggccgtcgctgcgtgggggagggacacggtaattcccgccctccctctcgcagcatcctggtccgatcccagcaaaattccacaggaagttctccgcatcatctccgattattacccggatgcgccaggggcgcgggcgcgtcggaacccgcttcacgtggaaggactagcgcttatgcgtgccagaaatcccgcccctctcgcattgctacttggtgacgactattcccattatcacactccccgcaatcggtccccaatgtgttgttgctggagtgctccgcggggagcaacgccagagtcacccagatgcaccggacacacgcgcaccaggcgcccaaggctatccgtctcctcagggaggggggaaactagaacccttgtacccagattagactcttcagaggataggcggtcgcgtcaacgccatagacgcatgactaactaccctctaccgcatcccactgtcactgacccttgggtggtaa

SEQ ID NO: 6 - DNA, 1245 bp, synthetic construct
  source 1..1245: mol_type = other DNA; note = gene gC
ttatgttgtcttccagcaccatgcgcccaagcaagcaactagtaacccacccacaaaagcgatactaattataataaaaatcgcagcgactgcggggaatccttgccgcattggcagattatctatcattaaggaggcgttgagatatctgtatgcatgttcatagcctcttatgacgcacacgtaattagaggcagattgaaatgtatcctccggtggtagttttatgacgtccattagtgtaacgatatttttcccctcgggctcatccaaaacaaaggtctggtgtgggctcgtcgaaattctattatctacgatccattcaataaatcctcggccgtctataatattagctgctctacacagggcataggcaggagggacgaattgtaccgtgatgcggggcttgctaatccagctgggagtcaaccttaacaagcgagtttcaggtcgtagcatttcaaagtcctcaggaggggtccatcgatatacaaattgaattttggaagggaggggctggctggcttctccacgtatttccagttcccatgtctgatccaccgatccattctctcctgatgcataaagaactcttctttctaaactgtatttttcagcaagagatgcattatgtatcatccatgtacccagcgcagcagatggaggataagcgccaagcgcttcaacacttaagtgcgaagccttatctgagtcttcgtacagttcttctgatttgatgcggacagttgtaggtggtcttgccaaaaagacgtcgataaaagagctatttagttttgtagaccctcgacttgcggtccactgataacgcgccatgtgagatctcatgctagacttgatagagacattgaaaaaacctgaacgacagtcgtccaaatatggtgggttaattccatcttcaccaatgtatcctaggtccttgattgaatcagtagcggcatcatatgtgtagagagttcttggagtagctctccatggatctagcgaaggggggatcccgtaggcgcttagcgcagtttcaactaagctggctgggtgtcgaattacttctacttttacagattcttcgtgggttagaggtctgtcgctgaagcattgcaatagaatggggtcaccgagctgccctcgaaaaacgtgtggcggatcacagaaaaattcaaacgcaaggctttgcaggctcaagagcaaaagtatactcgaaactagcgcagtactctgatgctgcat

SEQ ID NO: 7 - DNA, 708 bp, synthetic construct
  source 1..708: mol_type = other DNA; note = gene gL
ttatgggctgctcgcatagtccgggtgatagtggttgatgttcgtttccggccatctgaaatttatggtggtccttttatacaacgaatcaacaagtccacgagccaagacgttagcagccgcaagcccggtagcttccagccggtcttgaagaagtctaatccccaagtacggattcacttttgcagcgtacccattcttatagtagaaaacaacttccgtgtttacgcacagaggtctgtaaataatcacagtcaaattttcaaagtagtctcccgatggcaacggcccgaaaattcgcgcaccgttatctcgcgaattactatcacatggtgcggccgcggcagaaacaggatcacgaaaaatgccaggtatatcgacacttgaacatagcataagagatgctattagaaaaaaacaagtcaacattttaaaaatcattatcatatggagacctaggtctccgttcacgatccgtgttacgtcgacgagcagatggaactctttgcgcccgacttctaaatggatcgtcgtcagaatctgagtcgtccgaatacaaatgtagtggtctacaggaaccagtaggtactctttgtggcggacttttgaatggatcgtcgtccgaatcagagtcgtcccaatataaatatgacggcctagatggagtacttcggctctgtagtccggaaggaccttcacctgcgtcat

SEQ ID NO: 8 - DNA, 354 bp, synthetic construct
  source 1..354: mol_type = other DNA; note = gene gN
atgaggctgccgtgggaaaggagttttatgctgttgggagcagtggtgtatttgctgcatttggcactagtattcgcgcaaccgggtgtgacaccaggacacggaatggagcatgggtattatggtcttggcgctggaaatatgcttggagcggaaaattcgatgtatagcgcgaacatggaacacttttaccacagtgcctgctcgtcgcgcggattttccttagtaaacggaacagctgcttccgttttctttttcattagcctggcagtggcgttgattggcctgctggccgtgctatataacggttgtttcaacagctttaaaagcagtgtcattagttctcgatggtag

SEQ ID NO: 9 - DNA, 1090 bp, synthetic construct
  source 1..1090: mol_type = other DNA; note = gene TK
acgaacccatatcagcattgtagcgctctactgcgtcaattaagctcgcgatcgccgctcgtttggttgtaagagaacttgccgtctcggcaaactctaaacatttcagtgcacatgcctccggggtgaggccatgtgctggtaagtaaaaaatgcacaagtttttcaataccgtcgcgagtttttgaaccacaaatgaaagtacagtttttaatctttccccgtgaacattttgctgatcaaaagctaatattgcaaatagcgtctggtcgattgaaggattgatttctggaacagtagcagtttctaagcactctctcttgaattttaagagcgtggcacttgttaaggcttcttgctccttcaacaatgctggataagatgtacgttggaggtaggtggtagtattcatgaggcagatgtacgagttacgtaatgcacacaaatacgtccagtcgaccgtttcccctgggcgccccctttgtattatacgccgtgcctgttcgcaaatatccaaatccattaaaacaatgttggagtgtgttggaatgtcaggaattcctgacaataaagttattaaatctccaaattcgagcgctccgcttagatattgtgcaacagggaaacacaaacatgccgcgagtgggtgcctatctacgaggaataagacatcatctggtccggatgcagctgcgctgtagcagcgttcagatcccaaataattataaatgacgtggttcattattcttagtggggcagcaatagtcatctgaacttccgctacgcgacgagacgcctcccgacattcaagttttccggaagcctttagggtttgtatttcattaatttctttgatcatatccgctccgccaaaccattggcgccatgcttgcataggttccgctaagtatagacggcaacctctccaatttggcatttgcatcaacgtctttgcagtgactgttttaccgactccgaatggcccgtcgacgtaaagaagtacaacgcggcgaaagtcggagtcatcgttctcgaactcggagcaaaactgcacaccaccggaagttttcacggcgccagctacggccat

SEQ ID NO: 10 - DNA, 1182 bp, synthetic construct
  source 1..1182: mol_type = other DNA; note = gene gM
atgagcggctactactatagtggacaggaatcgcgtttagagaggattgcatggcgcatgtggctgattgaagtaatctcttttgtggttctggtgttgatatcactggcctcagctttctccacccttagtccgcaagcagggtttccctgttttttcggcaacgtcgccggtggacaccttaagccagcaacgcctttgatcaatcaacctggactgctaagggcgaacgaagatggacttttttttatttcgtcgccaggaatgatatttaccgtttacgcgactatggcggtgtggatactcttggcgatatacgttactcttggcgcagttagagccaagacttgtaatcacgatcgttcatacggagcgtccgaattgacttcggccgtgatcagcacacctgccttaatgtctattattatcacagtatgggcgtggcagttgtttgtactactcctttcgtacagacagcttacgctatcagctgtcacattttgcattatttttgtatccggcatcgtatttttgggtacgttcgcgtcttccggaaagacacctgaccagtactcagtttttaactcacagctcaaaagtgcatgccctgaagttcattgtgttgtatcaccgtttaaggcagtggtattaaacgtctacgcctctatcatgggagtttgggggcttataatggccttgtccggagcactcgtcatgaccgtgaattttggagtcagtgtccctgcttctacacttggggcaatgattgcatttcttatcataggtatcatatacttgattgtgaccgagctggttttagcgaagtatgtgcatgtacttattggtccgcatttcggactcatcattgctcttggaatgcttgggacggcatccctgcactatgctaatacgttggatagcggaactcatcccaactggaagccggctgcggcaggaattctaggagtatttgccctccttgttttagttttggctattctgcgagtcgtacgggcatacagatatcaccagtacagacagacggatttcatccgcaaggtaacgaccgttgccacagaagctttagataaaaccgtgaaacgcaaaacgcgccgcgaaaaatacaaaagccgaagagacaagcacgtttatgaaactgtaccctatgacgacgaactgtaa

SEQ ID NO: 11 - DNA, 1305 bp, synthetic construct
  source 1..1305: mol_type = other DNA; note = gene gD
atgcaccgtcctcatctcagacggcactcgcgttactacgcgaaaggagaggtgcttaacaaacacatggattgcggtggaaaacggtgctgctcaggcgcagctgtattcactcttttctggacttgtgtcaggattatgcgggagcatatctgctttgtacgcaacgctatggaccgccatttatttttgaggaatgctttttggactatcgtactgctttcttccttcgctagccagagcaccgccgccgtcacgtacgactacattttaggccgtcgcgcgctcgacgcgctaaccataccggcggttggcccgtataacagatacctcactagggtatcaagaggctgcgacgttgtcgagctcaacccgatttctaacgtggacgacatgatatcggcggccaaagaaaaagagaaggggggccctttcgaggcctccgtcgtctggttctacgtgattaagggcgacgacggcgaggacaagtactgtccaatctatagaaaagagtacagggaatgtggcgacgtacaactgctatctgaatgcgccgttcaatctgcacagatgtgggcagtggactatgttcctagcacccttgtatcgcgaaatggcgcgggactgactatattctcccccactgctgcgctctctggccaatacttgctgaccctgaaaatcgggagatttgcgcaaacagctctcgtaactctagaagttaacgatcgctgtttaaagatcgggtcgcagcttaactttttaccgtcgaaatgctggacaacagaacagtatcagactggatttcaaggcgaacacctttatccgatcgcagacaccaatacacgacacgcggacgacgtatatcggggatacgaagatattctgcagcgctggaataatttgctgaggaaaaagaatcctagcgcgccagaccctcgtccagatagcgtcccgcaagaaattcccgctgtaaccaagaaagcggaagggcgcaccccggacgcagaaagcagcgaaaagaaggcccctccagaagactcggaggacgacatgcaggcagaggcttctggagaaaatcctgccgccctccccgaagacgacgaagtccccgaggacaccgagcacgatgatccaaactcggatcctgactattacaatgacatgcccgccgtgatcccggtggaggagactactaaaagttctaatgccgtctccatgcccatattcgcggcgttcgtagcctgcgcggtcgcgctcgtggggctactggtttggagcatcgtaaaatgcgcgcgtagctaa

SEQ ID NO: 12 - DNA, 2620 bp, synthetic construct
  source 1..2620: mol_type = other DNA; note = gene gB
ttattcgtcttcgctttcttctgcccacgaagggtattcgagcagggggtagtcatcttccttggttcggggttttctgcgaaataggtttctcatatttttagcaaaagcagaaaaccgcgctttctgtcttcgagctttctgttcatccttgtgtagaagctcaaggcctttcagaactctggtggcaagtatcctgtcgtcctcatcactatcctcctcaacctcctcctcatccttgtagtaacgtctagagggtcgtggaggagttccggccgggggaactgcgcctgggaacagaacctgaactgggtttgatttcaggttcattacatatttgaacgccagcagtcctaaaataatgctcaccaccaccgctatcccaattccgagtgcggcgaacgggttagaaagaaatgaggcgatgccagatactgtagaaattactgctgccgcggctgtcatcactacagttcccaatgcctttcctacttctccaagagtgttgccaaaaaaatctgctattgctctaaagattgcatctcccctatctccacgtattacagtgtcaatgtctctgaaccttttgttataaatattttgatatctgaccacatcatcatagtttaaagtcccagtatctctgagttcctcgcgagtgtatacttcgacgggcacaaagtccaaatcttctagtatagtggcgttcaagttgatgaatatgctcacttcttcgatctcggaagcatttacttgtctaacaaatgtatagtcttcgtaaagaaggtagttttctcccaacagaaagtaccgtctgtgattgatcatgcatggttctatcaaattccgcccttgtaaaatttcattatgttctccgagttggcctaatatgtcaagattgtggttttctgttgagttcgcagaaaactgtgactcaggggaggacgaatacctgaaaataagtactggtctggtatagcacatggttgggtcccctggcatgcgcatggaatcctgcatcctaatattttcgattggaatttctatacattttgataccgctacgatgtctcccaatagcctggcgcttacaggttgtccgaacaaagatgtcatcagtgagttcgggtttagtttcttcatctcatgccatacaatcagttggcggttctgaagctcacaccacgcttccaacaaatttccgataagctcgttaacatgggcttggattttgtcgtatgcaaattgtaacatggcaaatgttgcagaactgtgggtagtaattctatcaccctgaggtccagactgcagcgacgccgtgcggcgacctgcggcttggcgacgctctctccctctcgggagatgattttgtctttgtgcctcttctaggtacatttcagccaagccatggctcatgagtttctgaaatgcgatcagaaatccgccactaccgagatagtattcgatgtccccagaacggacatgagtcgaactatactttctggcaaagatgccatctatggcctccgaggctatggtaggaacgcattcagccaaatgaagcctgctgatgttaaaaggttgttttccggacgagaacgtcatagtcatatctttaagtgaaaagtggtaactgtttttgtacgaaacacgaactgcttccgggacttcaatccatttactgagagtacatacagattccttttcttccattgcatcccagcctatagtgaattgttcatctgttagaaagtttctttttttagggggtcttatttgtccggtttccaaatccctgacttgataatttgcgatttcgagaaatctatagtctctgtagacactgtgacgccttggtccggtcgtgtttttggtataaaaaggagaaatttctactgtatcacctgtcgccattccaaagtaatcatacgggtatacagatctagcctgtagatattccacaacacagtcgaccgatgtagacgttcggtatcccagggtttgatgtcgcttagtaaagttagttgtatgaaacgccttggagacagttgatcttaacagtgatggaaccaggggcaattttttttctgcttcatcattgtcgtaagcttcaaaaaacatgaaatttttatgatacgttgctttggatgagcattctcccgatcgatcaatcctgacaatttcatgatagtctattggaaccctggtcacgtactcatttgttatttggggtcttgagaataatgcccacgtagtaactgtggttatatgtttatagtatagagtcacattaaagacgtacggggcaatgttttgcttgaagactacggcaattccttcagtcatgttagtcgaatcggcatgtcggtgacaatgtcgtggctgggcaagcctcgcaacactggcgccagttggagatgagcacgcaaaaattctgtttctaatctggggttctttcggtgcccccgacccaatggcttcggagaaagagatttttccaacatccatgctggctgtatcacgagggtctattattccagcaattccgtgtgaatcttgagatagggtagatgggatcaggattgccacgcacacgcagatcagcattttcaagctagcc

SEQ ID NO: 13 - DNA, 2415 bp, synthetic construct
  source 1..2415: mol_type = other DNA; note = gene gH
ttaaagttcgctctcctcgtcagacggtagccgtacacgtctaaattgcgttgccgagctgaggcagaatttatatatagccctgatggcactccatagagcaaccaatccaacaagtgcccctaccacaataccgataatgtatgccttgtcaaaaactattatgcttctgtcaagtcctaaaattcggacagcagttccgtgtggtcctagaagcgcgtagttatagaactcggggttcatagacttaaaaaatgcaatagatgagttggcaccggctaacagttctgattgaacatatgcactgtctataaagaaagcatcctggaacccccgtagaccgtattgaactatcaggtggttgcaatatatgcaagttttcgctcgtccattcaaatgaccatcctccatttttgctgttcgcgcgatttgcggtagggagttgcaacttgaattcacaaacgttacaaacaaaggggcagctgttgaggtatccggaagtctgtaggtcattccagtgctcggcgggctacttgtcatcacccaactagaatgcgcggtaacagggaggaaaaatacgatgtggtgttccaggtcccagtttaaacatttctttacctctggaaaaaacgcagttgccaggatctgaggggtcaacagagcttgtggcggtatcggtgcactatatgatggcaattcaccttcagatgataaatatattttggtgtaaaaaaatcgaaatggagcagatgctgtcagaattattttccatggtgcacttctggaaatatcaaaccttgtcgcaattgcacaaggagaaaaccactcttcaattttcgatgcctgtcctttcttgaagctcccgtctggttgggagtgaatctcgttgatatcggctagccggtcgacagagcataaaattgtcatgcgcctgaggaagttccttccatgttgtatagaaagttgaggagcccgcaaaataatgtacgtagaaactgccaataactttctgtacgcgggggtcattaccttttgtgtaatgatttctctgtaactgttatctattatttcaagagccagttggctgggggtcactccatcgatgctcgccaggaatgcattttggggatcttgttctgcagtgataagcaccgcattcatgatagtaagaagcaggttatgattttttatcatgggttcccatggcctgtcgtgcataattttaagaatactggatgggatagaagtaagagcaccaccgacattctgaataaagcgaatattcgttgtatagtttactgaattcgcccccccaaaacacactgcgacttggtctgcccatgtttgcgtttctgcaataaactcgacgatctccgaatcagttacgtatgcatcgatcgtaaactttagaaatcgaaaccacgcggccataaaccgtgctgcgaatcttacaaaaaaagactcgtctatggtattcgggcaacctgcgattgcgaccttctgcaaagttaaaaagtgtcccgcggatatttgttcgtcgaagctggccagctctaagaaatcttgagcagcggtcgttactgctgtaaacaaatttggccgataatttggtccagctgcgacgttgatgcgcacagttaacccctgatcttcagccttcagcatatagggccatgacttcagggacgtttctttaaccttcgcgctggttggtgtaattatcatctctatttgaggagaatgctgggtcgcgattataagggtcgccacattcagtccgatgttaatcttaagatctatgtcactagtaactgcgaggctaatatatcgtgtaaacacaaatggaaaaacagtttgtaaaggatcactgtaaattacacgaatcggagcgatgatgctctcgcccctatgaactgagttatttggtggaaagttgacgagtttcgcagatgtaagtgcattccccttgtatcctaccaaaagtcgatgctcaagctttggatcgttgctgaattttcgtggttggatggtagcctcgctagcagaaccccatctcgagtaagtataggctacgtacagttgtccaagcaaatctagagcgatcgataaattcctgttagcgcttccaggaggcccaaagattaagtttcgatcgacaaggcatagttgtgtgatgtggccgttttcgactgctgtcgctatcgcaatgaattctttcggaggggccaaattttcactggtggggggcacgaagacaatttttgcctttgccttgtcatcccctttagctctaagtaatatcgccagaatgaggaaaaatatttctctcgcgtgtcccatcggcgcgggttgctggtgaagccacgctctctcgagtaagaatgagtacaaagcaaggaaatgagtaaaggacat

SEQ ID NO: 14 - DNA, 1089 bp, synthetic construct
  source 1..1089: mol_type = other DNA; note = gene gI
tcacatttttattgagtcgggcgagcttagcgcagacgggttaacaatcgcaaccagttccacaagttcactatcctggtcagcccccttgtttatttcgcacgcaaccatgggtccaaattttcgcgattcccttctagtttgggaacgttcttcgtcgtcttgcgagaccgtgtctaattttcggtgcggggtgcacatgtttctggtgacgatggaaattacaatgacgaggccgatcgttgcgctcacgacggcagccacggttaccccaattagcgtagggctcattgtcccgagacggacagtaacgtttgaattttcgtttgcgggtgtcggttcgtaatgatccacgccattttctagccagggaaaggtaaagtgttccgcttcaagttcggaggcgctggttgcagtgacgggcgtcggggttgtcgcctcggcgctggtcgtcgtggcctccgtggcaacgtacgcttgccagttgcgccaactttcctcggtcctgagataggcctcaagttggacggtaggtaatccgaaggtgcgacacgaagccttggaatagagtccgcgagtcgcggcggtgttcgcgaattgatacaccgatagttggatcgcgaagacgtcaatgggttctttggtgtcgtctagccgaacaattacgtagaacatcccgctgtcgttcggacgaggatttcgtatagtgagcttaaacgggtgtggaggtccggttagtctgttcgaggcggagaacgtgtactcggtcactttacagccgacaaaaacagaagacgatcgatgagtgcaattttcaaagcgctcctcgctaagttcctggtagcactggtttgttatatcagaccgaaacgcgacgcggacggttcctgagtagggtttgtggggtctctggccaggcatgaaaaatagctgcagttgaattgtagcttcggggcgaggcgcgacgatcacgatgtgatcgtcgtcaatcctggcggagacgtgatttccagtgatcacgattcccatcgcgccgaagggtgcgagcgtcgcggcaaggagagccagagttccaagtagcgatgccat

SEQ ID NO: 15 - DNA, 20 bp, synthetic construct
  source 1..20: mol_type = other DNA; note = forward primer for gK
gcatacacaacggttgagac

SEQ ID NO: 16 - DNA, 20 bp, synthetic construct
  source 1..20: mol_type = other DNA; note = reverse primer for gK
ggaatctactcgccaggacg

SEQ ID NO: 17 - DNA, 20 bp, synthetic construct
  source 1..20: mol_type = other DNA; note = forward primer for gG
ctgaccgctagcccatacag

SEQ ID NO: 18 - DNA, 20 bp, synthetic construct
  source 1..20: mol_type = other DNA; note = reverse primer for gG
tccagtgcaagttcgtcctc

SEQ ID NO: 19 - DNA, 20 bp, synthetic construct
  source 1..20: mol_type = other DNA; note = forward primer for gE
cagtatcggacacggactgg

SEQ ID NO: 20 - DNA, 20 bp, synthetic construct
  source 1..20: mol_type = other DNA; note = reverse primer for gE
cgggttgactggaaacggta

SEQ ID NO: 21 - DNA, 20 bp, synthetic construct
  source 1..20: mol_type = other DNA; note = forward primer for gC
agactgtcgcctcgaaactg

SEQ ID NO: 22 - DNA, 20 bp, synthetic construct
  source 1..20: mol_type = other DNA; note = reverse primer for gC
ttggtcaacactgcaaagcg

SEQ ID NO: 23 - DNA, 20 bp, synthetic construct
  source 1..20: mol_type = other DNA; note = forward primer for gL
actgtaacagcgtctcaccg

SEQ ID NO: 24 - DNA, 20 bp, synthetic construct
  source 1..20: mol_type = other DNA; note = reverse primer for gL
taccggagacagaagagggg

SEQ ID NO: 25 - DNA, 20 bp, synthetic construct
  source 1..20: mol_type = other DNA; note = forward primer for gN
attgcaaggaatcgaggcca

SEQ ID NO: 26 - DNA, 20 bp, synthetic construct
  source 1..20: mol_type = other DNA; note = reverse primer for gN
tacttcttcgcctcctccga

SEQ ID NO: 27 - DNA, 20 bp, synthetic construct
  source 1..20: mol_type = other DNA; note = forward primer for gM
ccccacaaccatcgacagaa

SEQ ID NO: 28 - DNA, 20 bp, synthetic construct
  source 1..20: mol_type = other DNA; note = reverse primer for gM
gctgagatcgcatccgtaca

SEQ ID NO: 29 - DNA, 20 bp, synthetic construct
  source 1..20: mol_type = other DNA; note = forward primer for gD
taccgctggccaaagtacag

SEQ ID NO: 30 - DNA, 20 bp, synthetic construct
  source 1..20: mol_type = other DNA; note = reverse primer for gD
tagtctgttcgaggcggaga

SEQ ID NO: 31 - DNA, 21 bp, synthetic construct
  source 1..21: mol_type = other DNA; note = forward primer for gB
gacagtcgatgcttctatgcc

SEQ ID NO: 32 - DNA, 20 bp, synthetic construct
  source 1..20: mol_type = other DNA; note = reverse primer for gB
atgcaatcctacatcgccgt

SEQ ID NO: 33 - DNA, 20 bp, synthetic construct
  source 1..20: mol_type = other DNA; note = forward primer for gH
tgcttctttgacggagccat

SEQ ID NO: 34 - DNA, 20 bp, synthetic construct
  source 1..20: mol_type = other DNA; note = reverse primer for gH
ccgagacggcaagttctctt

SEQ ID NO: 35 - DNA, 20 bp, synthetic construct
  source 1..20: mol_type = other DNA; note = forward primer for gI
cctccagaagactcggagga

SEQ ID NO: 36 - DNA, 20 bp, synthetic construct
  source 1..20: mol_type = other DNA; note = reverse primer for gI
ccggaacgtttctgggatca

SEQ ID NO: 37 - DNA, 19 bp, synthetic construct
  source 1..19: mol_type = other DNA; note = forward primer for TK
catgactacaattttgccc

SEQ ID NO: 38 - DNA, 19 bp, synthetic construct
  source 1..19: mol_type = other DNA; note = reverse primer for TK
acgaacccatatcagcatt

SEQ ID NO: 39 - DNA, 20 bp, synthetic construct
  source 1..20: mol_type = other DNA; note = forward primer for UL32
ggtatgagatgtagcgatgg

SEQ ID NO: 40 - DNA, 23 bp, synthetic construct
  source 1..23: mol_type = other DNA; note = reverse primer for UL32
ggtagtttagtttagcatcggta

SEQ ID NO: 41 - DNA, 20 bp, synthetic construct
  source 1..20: mol_type = other DNA; note = forward primer for ICP41
tgcttttcctgcagcgtcta

SEQ ID NO: 42 - DNA, 20 bp, synthetic construct
  source 1..20: mol_type = other DNA; note = reverse primer for ICP41
tcttgtctcagaacgtgccc

SEQ ID NO: 43 - DNA, 17 bp, synthetic construct
  source 1..17: mol_type = other DNA; note = forward primer for ICP42
cgtgagtgaggaagggg

SEQ ID NO: 44 - DNA, 17 bp, synthetic construct
  source 1..17: mol_type = other DNA; note = reverse primer for IP42
ggtacagacgggtcggt

SEQ ID NO: 45 - DNA, 20 bp, synthetic construct
  source 1..20: mol_type = other DNA; note = forward primer for ICP43
ggcatcccgtactcttcgag

SEQ ID NO: 46 - DNA, 20 bp, synthetic construct
  source 1..20: mol_type = other DNA; note = reverse primer for IP43
tcactcgggtcttgttctgc

(19) United States
(12) Patent Application Publication
(10) Pub. No.: US 2026/0306619 A1
(43) Pub. Date: Oct. 8, 2026

(54) NATURAL ATTENUATED STRAIN OF INFECTIOUS LARYNGOTRACHEITIS VIRUS WITH GOOD SAFETY AND GOOD IMMUNOGENICITY AND USE THEREOF

(71) Applicant: INSTITUTE OF ANIMAL HUSBANDRY AND VETERINARY MEDICINE, FUJIAN ACADEMY OF AGRICULTURAL SCIENCES, Fuzhou, Fujian (CN)

(72) Inventors: Bo HOU, Fuzhou, Fujian (CN); Chenyan WANG, Fuzhou, Fujian (CN)

(21) Appl. No.: 19/106,818

(22) PCT Filed: Jun. 28, 2024
(86) PCT No.: PCT/CN2024/102158

§ 371 Date: Feb. 26, 2025

(30) Foreign Application Priority Data
Dec. 29, 2023 (CN) 202311854854.1

(51) Int. Cl. A61K 39/245; A61K 39/00; A61P 37/04

(52) U.S. Cl. CPC A61K 39/245; A61P 37/04; A61K 2039/5254; A61K 2039/54

(57) ABSTRACT

Provided is a natural attenuated strain of infectious laryngotracheitis virus attenuated strain (ITLV) with good safety and good immunogenicity. The natural attenuated strain, designated as ILTV FJ19, has been deposited in the China Center for Type Culture Collection on Nov. 29, 2023 with a deposit number of CCTCC NO: V2023109, the deposit address is No. 299, Bayi Road, Wuchang District, Wuhan City, Hubei Province. Experiments have shown that the avian infectious laryngotracheitis virus ILTV FJ19 strain has a stable passaging ability, may adapt to inoculation via the allantoic cavity route, has good safety and good immunogenicity when inoculated to 14-day-old SPF chickens. The strain may be used through eye-drop vaccination or oral administration, making it a candidate strain for attenuated live vaccine against ILTV.

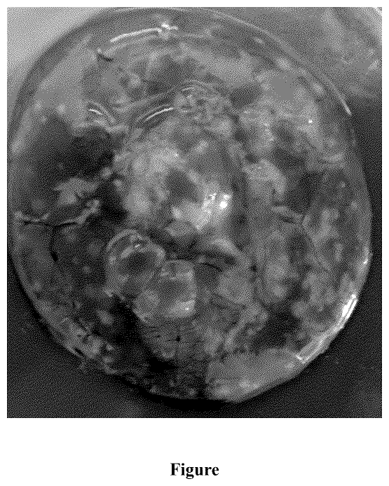

### CROSS REFERENCE TO RELATED APPLICATION

The present application is a U.S. national phase entry of PCT application No. PCT/CN2024/102158, filed on Jun. 28, 2024, which claims the priority of Chinese patent application No. CN 202311854854.1, filed with the China National Intellectual Property Administration on Dec. 29, 2023 and entitled “NATURAL ATTENUATED STRAIN OF INFECTIOUS LARYNGOTRACHEITIS VIRUS WITH GOOD SAFETY AND GOOD IMMUNOGENICITY AND USE THEREOF”, which is incorporated herein by reference in its entirety.

### REFERENCE TO SEQUENCE LISTING

A computer readable XML file entitled “GWPCTP20241108426 seqlist”, which was created on Jan. 23, 2025, with a file size of about 71,087 bytes, contains the sequence listing for this application, has been filed with this application, and is hereby incorporated by reference in its entirety.

### TECHNICAL FIELD

The present disclosure relates to isolation and identification of a natural attenuated strain of infectious laryngotracheitis virus (ILTV), and the determination of its immunogenicity and safety as a live vaccine, belonging to the technical field of microorganisms. The strain was isolated during conventional pathogen monitoring of a chicken flock in China. After isolation, identification, purification and cloning, the strain was obtained, designated as ILTV FJ19 strain. The strain was determined for its safety using 14-day-old specific pathogen free (SPF) chickens, and its immunogenicity was determined by immunizing 14-day-old SPF chickens with the strain and then challenging them 21 days later with WF03 strain (patent strain, patent no.: ZL202110385416.X). The determination results show that the infectious laryngotracheitis virus ILTV FJ19 strain of the present disclosure has good safety and immunogenicity, making it a candidate strain of an attenuated live vaccine against ILTV.

### BACKGROUND

Infectious laryngotracheitis virus (ILTV) belongs to the family Herpesviridae, genus Alphaherpesvirinae, which mainly cause death and/or decreased egg production in chickens. Severe infections are often manifested by dyspnea, gasping and expulsion of blood-tinged fluid, with a high mortality, while mild infections are primarily manifested by a variety of forms such as mucinous tracheitis, rhinitis, conjunctivitis, reluctance to move and low mortality rates. Both severe and mild infections cause serious losses and threats to poultry production.

ILTV has a genome size of about 155 Kbp, and is a double-stranded DNA virus. Like other herpes viruses, the genome contains structures such as a unique long region, a unique short region and inverted repeat sequences. It encodes a large number of glycoproteins and proteins involved in virulence and viral replication. ILTV wild strains vary greatly in virulence, where some strains exhibit high morbidity and mortality rates upon infection in chickens, classified as highly pathogenic strains, while others cause mild or insignificant symptoms and have no associated mortality, classified as attenuated strains.

Attenuated live vaccines are the primary type of vaccine to prevent infectious diseases in animals, but most of the current poultry vaccine strains are attenuated by avian embryos or cell passages. This method carries uncertainties regarding the potential reversion to virulence of the attenuated strains used for vaccine preparation. In contrast, natural attenuated strains exhibit advantages of low toxicity, fewer side effects, good immunogenicity, etc.

### SUMMARY

Embodiments of the present disclosure aim to provide a natural attenuated strain of infectious laryngotracheitis virus (ITLV) with good safety and good immunogenicity.

The technical solutions used in the present disclosure are as follows. The present disclosure provides a natural attenuated strain of ITLV with good safety and good immunogenicity, wherein the natural attenuated strain of ITLV is designated as ILTV FJ19, classified and designated as infectious laryngotracheitis virus, deposited in the China Center for Type Culture Collection on Nov. 29, 2023 with a deposit number of CCTCC NO: V2023109, the deposit address: Wuhan University, Wuhan, China.

The present disclosure provides use of the natural attenuated strain of ITLV described in the technical solution above in the preparation of a vaccine, where the vaccine is an attenuated live vaccine; the attenuated live vaccine is used to prevent infectious laryngotracheitis; and the infectious laryngotracheitis is an avian infectious laryngotracheitis.

The present disclosure provides an infectious laryngotracheitis vaccine comprising a live virus of the natural attenuated strain of ITLV described above; the method for administering the infectious laryngotracheitis vaccine includes oral administration or eye-drop vaccination; the infectious laryngotracheitis vaccine is used for vaccinating chickens, and the immunoprotection rate can reach 100% after 21 days of the vaccination followed by challenge with an ILTV virulent strain WF03 (patient strain, patent No. ZL202110385416.X).

The present disclosure provides a method for preparing the infectious laryngotracheitis vaccine described in the technical solution above, comprising inoculating an SPF chick embryo with the natural attenuated strain of ITLV as a vaccine producing strain via an allantoic cavity route, harvesting the infected embryonic body and/or chorioallantoic membrane, and performing grinding to prepare a viral liquid, such that the infectious laryngotracheitis vaccine is obtained.

The present disclosure is characterized by the following significant advantages. In the present disclosure, by isolating pathogens from a conventional inspection chicken flock, and identifying, purifying and cloning them, an ILTV strain that is genetically stable and can adapt to inoculation via a chick embryo allantoic cavity route is obtained, designated as FJ19 strain. Inoculation of 14-day-old SPF chickens with the P1-generation culture of ILTV FJ19 strain has demonstrated good safety, and good immunoprotection against challenging with WF03 strain. Inoculation of 14-day-old SPF chickens with the virus culture, which is obtained by adaptation of ILTV FJ19 strain to inoculation via the allantoic cavity, has demonstrated good safety, and good immunoprotection against challenging with WF03 strain. Therefore, the isolated ILTV FJ19 strain of the present disclosure has a stable passaging ability, may adapt to inoculation via the allantoic cavity, and exhibits good safety and immunogenicity when used to immunize 14-day-old SPF chickens. Furthermore, it can be administered through eye-drop vaccination or oral administration. This strain may be considered a candidate for an attenuated live vaccine against ILTV.

### BRIEF DESCRIPTION OF THE DRAWINGS

To describe the embodiments of the present disclosure or in the conventional technology more clearly, the accompanying drawings required for the examples will be briefly described below.

The FIGURE illustrates the pathological change in chorioallantoic membrane 7 days after inoculation of 10-day-old SPF chick embryo with suspected ILTV via chorioallantoic membrane route, showing off-white spots.

### DETAILED DESCRIPTION OF THE EMBODIMENTS

To make the content of the present disclosure easier to understand, the technical solutions described in the present disclosure will be further described below with reference to the specific embodiments, but the present disclosure is not limited thereto.

### Example 1: Isolation, Identification, Purification and Cloning of Strain

During conventional pathogen monitoring of a chicken flock, a positive tissue sample suspected of ILTV infection was taken and added to PBS (0.01 M, pH 7.2 to 7.4) at a ratio of 1:5 m/v. After ground, the tissue was frozen and thawed 3 times, centrifuged at 12000 r/min for 10 min, and filtered using a 0.45 μm filter membrane. A 10-day-old SPF chick embryo was inoculated with the filtrate via the chorioallantoic membrane route, and continued to be hatched for 7 d. The chorioallantoic membrane was aseptically harvested, and added to PBS (0.01 M, pH 7.2 to 7.4) at a ratio of 1:5 m/v. After ground, the chorioallantoic membrane was frozen and thawed 3 times, and centrifuged. A 10-day-old SPF chick embryo was inoculated with the resulting supernatant via the chorioallantoic membrane route. After two passages, the pathological change in the chick embryo was observed. It was found that after three blind passages, the chorioallantoic membrane of the chick embryo showed off-white spots (See the FIGURE), suggesting a suspected infection with ILTV. According to the Agricultural Standard in the People's Republic of China NY/T 556-2020, Diagnostic Techniques for Avian Infectious Laryngotracheitis, the strain was identified as ILTV.

The identified ILTV strain was purified and cloned using the limited dilution method. Specifically, Ten-day-old chick embryos were inoculated with the dilutions via the chorioallantoic membrane route. The virus liquid was diluted 10², 10³, 10⁴, 10⁵ and 10⁶-fold using 10-fold serial dilution method, which were each inoculated to six 10-day-old SPF chick embryos. The chick embryos continued to be hatched for 7 d, the chorioallantoic membranes were then examined, and judged as positive by the presence of off-white spots on the allantoic membranes. The allantoic membrane that tested positive at the highest virus dilution was ground to prepare a virus liquid, and the purification and cloning processes were performed 4 times in the same way. The two purified and cloned ILTV strains, designated as FJ19 strain and FJ22 strain, respectively, were subjected to scale-up culture in 10-day-old SPF chick embryos, and the chorioallantoic membranes were harvested to prepare virus liquids, which were designated as Passage 1-generation (P1 generation).

The virus liquid preserved at −70° C. was taken, thawed and equilibrated to room temperature. 0.5 mL of the virus liquid was added to corresponding 4.5 mL of PBS (0.01 M, pH 7.2 to 7.4), and the resulting virus liquid was diluted in a 10-fold serial dilution manner until the virus liquid was diluted 10⁶ times. 10-day-old SPF chick embryos were inoculated with the virus liquids diluted different times, with the virus liquid at each dilution for 6 chick embryos. Another 6 chick embryos were taken as negative controls. All chick embryos were placed in an incubator at 37° C. and continued to be hatched for 7 days. The chorioallantoic membranes were judged as positive by the presence of off-white spots on the chorioallantoic membranes. Based on the number of the positive chorioallantoic membranes, the content of ILTV was calculated as 10^{5.70} EID₅₀/ml according to the Reed-Muench method.

### Example 2: Culture Characteristics of ILTV FJ19 Strain and ILTV FJ22 Strain

The ILTV FJ19 strain and the ILTV FJ22 strain were cultured in 10-day-old SPF chick embryos. Specifically, chick embryos were inoculated with the harvested P1-generation via the chorioallantoic membrane route, and the virus liquids prepared from the harvested chorioallantoic membranes had the highest virus content. 7 days after inoculation of the chick embryos, large off-white lesions appeared on the chorioallantoic membranes or a large number of off-white spots appeared on the chorioallantoic membranes. The harvested virus liquids were detected according to the bacterium-free, mycoplasma-free and exogenous virus-free test methods from Chinese Veterinary Pharmacopoeia (2020 edition). The results showed that the virus liquids of the ILTV FJ19 strain and ILTV FJ22 strain were pure ILTVs that were free of bacterium contamination, mycoplasma contamination and exogenous virus contamination.

### Example 3: Determination of Immunogenicity of ILTV FJ19 Strain and ILTV FJ22 Strain

Sixty 14-day-old SPF chickens were divided into six groups, namely groups G1, G2, G3, G4, G5 and G6, where the chickens in groups G1 and G2 were not inoculated, the chickens in groups G3 and G4 were inoculated with the virus of the ILTV FJ19 clone (P1-generation) via eye-drop inoculation and oral inoculation, respectively. The chickens in groups G5 and G6 were inoculated with the virus of the ILTV FJ22 clone (P1-generation) via eye-drop inoculation and oral inoculation, respectively, where the inoculation doses were all 10^{3.0} EID₅₀ per chicken. Observations were performed for 21 consecutive days after the inoculation. After 21 days, the chickens in groups G2, G3, G4, G5 and G6 were subjected to intratracheal challenge with the virulent strain ILTV WF03 at a challenge dose of 0.2 mL/chicken, with a virus content of 10^{4.0} EID₅₀. Group G1 including non-challenged chickens was used as a negative control group. The clinical symptoms were observed for 14 consecutive days after the challenge.

The results showed that there were no visible side effects such as lacrimation, ophthalmia and respiratory tract response in the chickens of the inoculation groups within 21 days after the inoculation, and the typical clinical symptoms were not observed in the chickens in group G1 within the 14-day observation period after the challenge. The chickens in group G2 had morbidity morbidity of 100% (10/10) and a mortality of 40% ( 4/10), showing typically clinical symptoms such as hemoptysis and mouth breathing; The chickens in group G3 (eye-drop inoculation with ILTV FJ19) and group G4 (oral inoculation with ILTV FJ19) had morbidity of 0% (0/10) and a mortality of 0% (0/10), with no typical clinical symptoms. Unfortunately, the chickens of group G5 (eye-drop inoculation with ILTV FJ22) had the morbidity of 60% ( 6/10) and the mortality of 30% ( 3/10), and showed typical clinical symptoms such as hemoptysis and mouth breathing The chickens of group G6 (oral inoculation with ILTV FJ22) had the morbidity of 70% ( 7/10) and the mortality of 30% ( 3/10), and the infected chickens showed typical clinical symptoms such as hemoptysis and mouth breathing. The results showed that the ILTV FJ19 clone may be used for vaccination due to its excellent immunogenicity, while the ILTV FJ22 is not suitable for use as a vaccine strain candidate due to its poor protective efficiency against challenge.

### Example 4: Determination of Safety of ILTV FJ19 Strain

Thirty 14-day-old SPF chickens were divided into three groups, namely groups G1, G2 and G3. The chickens in group G1 were not inoculated, the chickens in group G2 were inoculated with the virus of the ILTV FJ19 clone (P1-generation) via eye-drop inoculation at an administration dose of 10^{4.0} EID₅₀ per chicken, and the chickens in group G3 were inoculated with the virus of the ILTV FJ19 clone (P1-generation) via eye-drop inoculation at a dose of 10^{5.0} EID₅₀ per chicken. Observations were performed for 14 consecutive days after the inoculation.

The results showed that the chickens in group G1 had no typical clinical symptoms, and also no visible side effects such as lacrimation, ophthalmia and respiratory tract response were observed in the inoculated chickens in group G2 after 14 days of the inoculation. Unfortunately, 20% of the inoculated chickens in group G3 suffered visible transient side effects such as lacrimation and ophthalmia at 4 to 6 days after the inoculation.

### Example 5: Determination of Safety and Immunogenicity of Virus Obtained after Adaptation of ILTV FJ19 Strain to Inoculation Via Allantoic Cavity

ILTV FJ19 strain of P1-generation virus was continuously passaged using 10-day-old SPF chick embryos via the allantoic cavity, and hatched for 5 to 7 days. Then the chorioallantoic membranes and the embryonic bodies or a mixture of both were harvested, respectively. After the grinding process, an appropriate amount of PBS (0.01 M, pH 7.2 to 7.4) was added, and the resulting mixtures were subpackaged and stored at −70° C. The EID₅₀ was determined. Allantoic cavity inoculation and passage were performed to prepare different generations of virus liquids, such that P2-generation, P3-generation, P4-generation virus liquids to P7-generation virus liquid were obtained.

Fifty 14-day-old SPF chickens were divided into five groups, namely groups G1, G2, G3, G4 and G5. The chickens in group G1 were not inoculated. The chickens in groups G2 and G3 were vaccinated by eye-drop route with the chorioallantoic membrane virus and embryonic body virus of the P5-generation ILTV FJ19 clone, which had been adapted to inoculation via the allantoic cavity route. The chickens in groups G4 and G5 were vaccinated by eye-drop route with the chorioallantoic membrane virus and the embryonic body virus harvested from the P7-generation ILTV FJ19 clone, which had been adapted to inoculation via the allantoic cavity. The doses for all birds were 10^{4.0} EID₅₀ per chicken. Observations were performed for 14 consecutive days after the inoculation.

The results showed that the chickens in group G1 had no visible clinical symptoms, and the vaccinated chickens in groups G2, G3, G4 and G5 did not suffer visible side effects such as lacrimation, ophthalmia and respiratory tract response after 14 days of the vaccination.

Sixty 14-day-old SPF chickens were divided into six groups, namely groups G1, G2, G3, G4, G5 and G6. The chickens in groups G1 and G2 were not inoculated. The chickens in groups G3 and G4 were vaccinated ocularly with the chorioallantoic membrane virus and embryonic body virus, respectively, of the P5-generation of FJ19 clone, which had been adapted to passage via an allantoic cavity route. The chickens in groups G5 and G6 were vaccinated ocularly with the chorioallantoic membrane virus and embryonic body virus, respectively, of the P7-generation of FJ19 clone, which had been adapted to passage via the allantoic cavity route. The doses were all 10^{3.0} EID₅₀ per chicken. 21 days after the vaccination, the chickens in groups G2, G3, G4, G5 and G6 were subjected to intratracheal challenge with the virulent strain ILTV WF03 at a dose of 0.2 mL/chicken (10^{4.0} EID₅₀). The chickens in group G1 with no challenge were set as a negative control. Clinical symptoms were observed for 14 consecutive days after the challenge.

The results showed that typical clinical symptoms were not observed in the chickens of group G1 within the 14-day observation period after the challenge; the chickens of group G2 had the morbidity of 100% (10/10) and the mortality of 40% ( 4/10), with the infected chickens showing typical clinical symptoms such as hemoptysis and mouth breathing; the chickens in group G3 (the chorioallantoic membrane virus of the P5-generation) and group G4 (the embryonic body virus of the P5-generation) had the morbidity of 0% (0/10) and mortality of 0% (0/10), with no typical clinical symptoms; and the chickens in group G5 (the chorioallantoic membrane virus of the P7-generation) and group G6 (the embryonic body virus of the P7-generation) had morbidity of 0% (0/10) and mortality of 0% (0/10), with no typical clinical symptoms.

### Example 6: Sequencing of Major Immunogenicity Genes and Virulence Genes of ILTV FJ19 Strain

ILTV virulence-associated genes gE/gI, TK, ICP4, gC and gG, immunogenicity genes gB, UL32, gD and gC, and viral replication-associated genes gB, gD, gE, gI, gH, gL, gK, gM and gN were subjected to PCR amplification, cloning and sequencing using the primers in Table 1, with the P1-generation virus liquid of ILTV FJ19 strain as genome template. The amplification system was as follows:

| 1-5 TM 2 × High Fidelity master Mix (MCLAB) | 25 | L |
|---|---|---|
| Prime F/R | 1 uL each | |
| Viral DNA template | 2 | uL |
| ddH₂O | 21 | uL |

TABLE 1
Names and sequences of primers for PCR amplification

| Name | Prime F | Prime R | Size (bp) |
|---|---|---|---|
| gK | SEQ ID NO. 15: | SEQ ID NO. 16: | 2216 |
| | GCATACACAACGGTTGAGAC | GGAATCTACTCGCCAGGACG | |
| gG | SEQ ID NO. 17: | SEQ ID NO. 18: | 1416 |
| | CTGACCGCTAGCCCATACAG | TCCAGTGCAAGTTCGTCCTC | |
| gE | SEQ ID NO. 19: | SEQ ID NO. 20: | 1894 |
| | CAGTATCGGACACGGACTGG | CGGGTTGACTGGAAACGGTA | |
| gC | SEQ ID NO. 21: | SEQ ID NO. 22: | 1983 |
| | AGACTGTCGCCTCGAAACTG | TTGGTCAACACTGCAAAGCG | |
| gL | SEQ ID NO. 23: | SEQ ID NO. 24: | 1377 |
| | ACTGTAACAGCGTCTCACCG | TACCGGAGACAGAAGAGGGG | |
| gN | SEQ ID NO. 25: | SEQ ID NO. 26: | 692 |
| | ATTGCAAGGAATCGAGGCCA | TACTTCTTCGCCTCCTCCGA | |
| gM | SEQ ID NO. 27: | SEQ ID NO. 28: | 1722 |
| | CCCCACAACCATCGACAGAA | GCTGAGATCGCATCCGTACA | |
| gD | SEQ ID NO. 29: | SEQ ID NO. 30: | 1850 |
| | TACCGCTGGCCAAAGTACAG | TAGTCTGTTCGAGGCGGAGA | |
| gB | SEQ ID NO. 31: | SEQ ID NO. 32: | 2799 |
| | GACAGTCGATGCTTCTATGCC | ATGCAATCCTACATCGCCGT | |
| gH | SEQ ID NO. 33: | SEQ ID NO. 34: | 2617 |
| | TGCTTCTTTGACGGAGCCAT | CCGAGACGGCAAGTTCTCTT | |
| gI | SEQ ID NO. 35: | SEQ ID NO. 36: | 1789 |
| | CCTCCAGAAGACTCGGAGGA | CCGGAACGTTTCTGGGATCA | |
| TK | SEQ ID NO. 37: | SEQ ID NO. 38: | 1251 |
| | CATGACTACAATTTTGCCC | ACGAACCCATATCAGCATT | |
| UL32 | SEQ ID NO. 39: | SEQ ID NO. 40: | 2023 |
| | GGTATGAGATGTAGCGATGG | GGTAGTTTAGTTTAGCATCGGTA | |
| ICP4₁ | SEQ ID NO. 41: | SEQ ID NO. 42: | 2244 |
| | TGCTTTTCCTGCAGCGTCTA | TCTTGTCTCAGAACGTGCCC | |
| ICP4₂ | SEQ ID NO. 43: | SEQ ID NO. 44: | 2668 |
| | CGTGAGTGAGGAAGGGG | GGTACAGACGGGTCGGT | |
| ICP4₃ | SEQ ID NO. 45: | SEQ ID NO. 46: | 1272 |
| | GGCATCCCGTACTCTTCGAG | TCACTCGGGTCTTGTTCTGC | |

The purified PCR product was inserted into a blunt end vector (pClone007 Blunt Simple Vector Kit, Beijing Tsingke Biotech Co., Ltd.). Competent cells DH5a were transformed, and then coated on ampicillin-containing plates. Five clones (bacterial liquids) per gene were screened for sequencing by Sangon Biotech (Shanghai) Co., Ltd., redundant sequences upstream and downstream of the gene sequence of interest in the sequenced sequence were deleted, and analysis and alignment were then performed. The sequence information of ILTV FJ19 genes gK, gG, gE, UL32, ICP4, gC, gL, gN, TK, gM, gD, gB, gH and gI are as shown in SEQ ID NOs: 1 to 14, respectively. ILTV FJ19 gene sequences were different to varying degrees compared with attenuated strains at home and abroad, including A20 (NCBI accession number: JN596963), K317 (NCBI accession number: JX458824), LT Blen (NCBI accession number: JQ083493), Laryngo Vac (NCBI accession number: JQ083494), Poulvac ILT (R) (NCBI accession number: KP677882), Rus/Ck/Tatarstan/2009/1643 (NCBI accession number: MF405079), Nobilis Laringovac (R) (NCBI accession number: KP677881).

SEQ ID NO. 1: gK (1020 bp)
TCAAGGCCCGAACAATTTGTTTTGTATCTTCTGTTCGTATTTAAATGCAATTATTACAAT
GCTCGCAATCGCAGCCACGCACAATGCACGCAAGACTAAGCTCGAAGCAATATTGGC
AAGGCATGAAGTCAAGATACTGGGAGGTTTCGATGCGGAGACTTTAGTCTTTGCCGG
AGTCGTGTAAGGGATCAGTGATAAGAGTTCTAGAACCGCTGAGACAATTACATATACC
CAAGCACTAATTTTTACGTGTTCATGTAAAATATATGCACACGGATCATAGCAAATCCG
CAATGTCATGGTGGACATAATAAGCGATGCTAAATATAAACCGATCTCGAGTGTTTTCA
GTAGTAAGACCCCTGGATTACAGACGCAAAATCCTAGAGGGTCTGCTACGTAGTTCA
AGCTTTTGCAATCTCGGAGATGTTCAAATTCCCTCAAAAACCGTTGCATCTTTGTGTAT
GGCACCCGTAATATCAGCCTCGAAGTAAACGCTGTCCAGTAGTTAAGATGATATGCAT
CCATCGCAATGCGTTCTCGCGGCAGACATAGCCCGAACATTCGCCTCTGGCGACGGG
TCAGCAAGTAACATATGTACAATACCGTCGAAATGAATGCAACTCTGATCTGGGAAAA
CCATATGTAAAGCAGACAGTCTGCCACTTCCATGACGAATACTGTTCTTTGGCCGGTG
GGATCAAGACGAGAAATGAGTACAGTCTCATTTTCAAAATAAGCTCCTTTAACACATC
TCGTCTGGTGGTTCTCGTAGTTGTTTAGACGCTGTGTATATTTTGTGAGGACATAAACT
AGTGAAGAATTAAATGTAGCGGGGGGTTGGTGAACAGGATTGAATTCTTGCTGTCCG
GGCAGGAGCCATTTAATATTTGCGAGTACGTACAAGCAGTCGTCCTTTGAGTCAGTGG
AAATTTTAGAGCATACATACCATACCAAGAAGACTATATGTATCGTTCCGGTTAAAATT
ACTGCCCATTTTAGACATTCTGGTCGCAGCAT.
SEQ ID NO. 2: gG (879 bp)
CTACTGCTGGAGCGTAGAGGGCGGTGAAAAGACAACCATCCTGGGCATCGTCTCTGA
CCTTTCGTCGACGTACTCGTCCAGCGGACCGGCGCATTCCTCGTCCCATAGGTAAAGC
TCGTCCGTGGCGTTTTCCTGCTCCTCGACGTCATGCGAACAAACGGTTGTCCATGGGC
GTAGCGGGGGCATGTCTAGACAATTGGAATAATCTTCCTCCATAGGACCGTCGAGTTC
GGGCGAGGCTACGTCATAAGATGATAAGTCTGACGCCTCGTCGGCCAGGCTGTAATA
GGTGTACCCATACTGGTCGTGGCATCCGGGGTTTGGTCCGACGTCTAGATAAACAGAC
CCGGTATATCCCTTCCCACCCACTGAGAGCTGGTAGGCGTAGATGCCGGCATCGTACA
TCCCTGGGTACAGCACCAGGGATCCATTGCCAGACACGTAACTACCAAGTTCCACCC
CGGAGCGCAGTCTTCTCTGCACTTTTTCGCACTGCTTGTCGAAATCAGGTATCCCGGC
GTCAGACGCCCTRCACACAGACGCGTTCAGATTATGGATTAGAGCCCAGCACGTAGA
CAGGTCCTCGTGTCCACTGCCCGTTTCGCTATCCACCCCGAACACGAAGCTTTCGGG
ATTACTATTCCCGTAGAACCATTTCACCTCGACTGACACTCCTTCGGTGCGACCAGAG
TAGTCGCTCCACGGGACCGTCACCTCAATCGTGGTGCGGTTGCACGCCTGGGCTATA
ATTTTGCCCCCGAACGGGAAAGGGCTCGACTCGAGCCCGTCCAGTACCGGCGCCCCT
AATACAGATGCGCACAAAAGCGAGCATACTAGGGAAACGGTGGCAATCGATCCTATG
TTACTGAAGCCGCTCAT.
SEQ ID NO. 3: gE (1500 bp)
CTAATAGCTATCCATATTCCTTCTGCCGCTTTTATTTGCCAGGATGCGATCTGCCATTGA
TGCGATGGCGGATTTAAAGTTTAAGGCCCTTTCTCCCTCCACGACCGCACCGAAAAA
GGTATTCACATCTTCGCGGAATTCCTGCTTACAGTAGCCGCGAAATAGGGGTTCGTATA
CTTCGTCATAAATCACTTCCTCATCGTCCGTAAGCCCGCCGGCCTCAAACTCGTTTTTG
GGGAGCGGCGCGTACAATGGTTCGGTCTTGGAAAAAGTACCCATTTGGTGCGCGGGG
CACTTCTTTTTCCCCACAAGCAGAGCGCATGTAAGAATTACGCACGTCGCGGTCACCC
CCGCGACGACGAAGTACATTTTATAGTTACCCGTTTCGTCCATTGGTCCGGGACTGCC
AATTACTACTTTGTTCATCGGGACCTTCGAGGGCATGTGCGTTTCGACTACTACGCGC
GGACTGTTACGTTTCCCGAGCACCGTGTAGCTCCATCCCAAAATGTCTCCGTCGGAAA
CGACTATAATCAAAACTTTGGGGATGGCTATAAGAGGACTGATCACCAGGCCTGCGCG
TGACTCGGAGAGTCGCGAATCGAACCGTAGGTTCGGCACGCGATTTTCGTGGCAGTC
TTCTAGAGTATCGCGGGAACAATCCAGCAAAGAGGCCGCGCCCAAGAATTCCGCCAT
CAAGACAGATCCTATGAGGCATCTCTGGTTGGTTGCGGGAAACTGACATTTGCCGTCT
GTAGGATGGTATACGCACGGCTCGAACAGTTTCACTTCTTCGCACTCCTCTTTGACAT
CAAACCCATACCACGAATACGTAACGTTAAATTTTACGTGCTCGGTCGGTGGGAAGAC
TCTAAGAACAGGGGTCACATAGGTGGGGCTGTTGTCGTTGAGTTCATGAAACGGCAG
CATTTGTAATTCCACGTGCGCGTGGGCCTCTGACGGCTTGATGGTGTCTATCTCCACCT
CGTGCGGTGCTGGAGCCTTCTCGTCTACGTGCGAAAGAACGCTGACGTTGTACGATG
TAGTTTCCATGGCCAGATCTGGCTGAGAGATCCGTCGAAAATAGATTCCCTCGTCCGA
ACGAGTTACAGGATCAAAGAGCAGGCCTCCGTACTCTGTCCACATGATATTAGTTATAT
TGCTGTTGTGTAGCCCAGAACCCAGCACGACGAAGCTTTCGCCGCGCTTTAGTAAAA
TTACCAGGTTTGCAGACAGGGGTCCCGCGGGCGGGCAGTCTTTCTTTCCTCCGTCGT
AAATTAAATCATGAAAGCAATAGTTAGGCGAAGAGCAGACATCGGCCGCCTTCATCTT
GCACGCGTTCCGCAGGACTTTTGTCCATTTGATTACAGTCCCTTCCGGAACGTTTCTG
GGATCATCTTCCCCGAGGACAGCCTGAGTGCCTTCCAAAAAGAGGACGTGTCGCGTC
GCAAAAGTTAGGCGCGCAAGACAAGAGGCGAGAACTATCACTAACATGTTCAT.
SEQ ID NO. 4: UL32 (1749 bp)
ATGACATCCAAACGATCAACTGCAGAAACTCGCACTGAACTTTCCAAGGGATGGAAA
GCTGGAGTGCTCTCAAAACTATATACTGGCTACGACCCGGCGATTCTGACGTTTAATG
ACAAGTTGTCGGACGAACTATTGTATGGAGCTCACCTAATTGAAATATATCATGAAGA
AAGTACAGAACAAGCTGAAGAACAAAACAGAATTCCGGACATTGATTCTTTCGTAGA
AACCATGGCTGACTTATACACCCTAGATAAATCATGCGCCGTCTGCAAGATTATTACCA
AATATCAAAAAAAATTCCCCGTGACTCCCGAATGGATGGTGGACTACTCTTTATTGTG
CTTCAAATCTCAAAGCATCCCCCTGTGTGCTACATCTACCTTCATAACTGCTTTTGAGT
TCATCTTCATCATGGACAAACACTACTTGCATTCCCACAGCACCTCTTTAGTTGGCGCT
ATAAGCCGTCGGGAATTTTCCCTCGGGGATATTCAGAAGCACTTCTTTTTTAACGGCT
GTTTCAAAGCGGTGGAAGGGGGACTGGACTCTAAAATAGATCTAAATAATTATTCGTT
CCTAGTGCAGTCGGTAGCACGCTATGCGTTACTATCTACTAGATATCAAGAAGCACTTA
GGGCAAAAATTGCCCCGGTCTCTGGCGCGTCTGCAGATTTCAACTTCCAAAAAAGTA
GCGGAATGGCCGTGGCTTTGTTAAAATGGCGTGAGTATGCCAGACCACTCGAGTGTT
TTTGCTCCGCGCAGTGTATGTTAAAGAGGAAAAATACAGAGACTGCCTCCTTCGCTTT
TCGAACCTGTGCGCAAAAAGCTGCTTTAGAAGATATTAGCACGGAGTCAAAATATGT
GGAAGGGGGACCAAGCGAAACCCTGACAACCCCAAGTAAATGGGGATTTACCGACC
TGACGGCATTATTAATTGCTGGAACAGCCGGTATGCCAAATCGGGAACTCGATGCCAG
CTGCAGCGACGAGCCTGAGTATGATGATACAAATGATAATGGAGAAAATTTCGTTGCC
CCTATAATTCGGACAAGAAATACCGTAGTGGAGAACTTTCACGCAACGCTAATCCAAC
AGGTCAAAAATACTAGGCTCCCCAAGACAGCGAAGCAGTACATTTGCGACAATACCA
AACCATCACTTGCCATTGGCCCAATTTTGGCGTCTATTTTGCCTCACCCGACTAGCCG
AGGAGATACTGGAGGGGAATGCGTGTTGTGCAATCTCATGCTTACTCGGGAACACTG
GCATGCTCTTCGAAAGCTGAAAAGTAAAGTAGTCGGCTGCAGTAACGTAAATAGCAG
CTTGTTCGATGGGATTGAACCAGCATTAGAAACGTTCGAAGACTATACTGCGTTAAAC
GATGGGGGGCGAATGTTAACCTTATTGAGGATGGCCGGGGCAAACGCAATTTACAAA
CACTTTTTTTGCGATCCATTGTGTGCAGTTAATACGCTTCGCGTAAATCCAAAAGTTTT
GTGGGAAGGCCAACCCAAGGACCCAGAACTGCTGCAGCTTTATAAAGCCGAAATCG
CCACGGCTAACATATTTCAAGAACGGGTTTGCCGCGGGCTGTGGATTCTAGCCTTCAC
ATTTAAAGCTTACCAGCTTTCTCCTCCTCGCCCTACCGCTTTAAATTCTTTCATTCGCG
GCGCTGAGACCTATTTAAAGAGGCACGGAATCAGCTGCATTGCTTTAGAACACGCAC
TGACTCGATATGTCTGA.
SEQ ID NO. 5: ICP4 (4450 bp)
ATGTCCTCTGATTCCTCGACGCCGAGTAATTTGGCCGCTCCAATTGTTGATGAGTCGG
GATTCTCAACTGGCCCGCCCTCAGATCTATTTACCTTTTTGCGCGCCGATAAGAGTGG
GCGCTACGATTCTTTGGGTTCTCCCATCCCGAACCCTCCAAACGCACCTACCTGTGAG
ACCACTACTGATAGCTTTTCGTACAGCACGCCACCTCTTTTTTCCAGCGAGGACGAGC
TCTTTCACTCTGTCATCGCTGAGAATGCGGCCCAAGACGTGGACGGGCGAGAAATGT
CTCTCCGCGACCTTATAGCGATGTTAAACGACATGGATCCCGAAAACATAAACTCTAT
AGAACCGTGTCCGAACGCTACCAGTGATGTCGTGGCACTAGATATTAACGTGGATACC
GATCAGCCCGGAGTTTATACAGTCATCGACTCGCTCGCACATCCCTGCGAAACTCCCA
TGGAGCAGCAGCACGTTGAATGCCCGTGTCCATTACCTGCAGTAGAGAAAACAACCG
CTGTGAGGCGTCAAGAAGTGGTGAGGAAGTCAGAGAGAGTGGCCAGATCTCGTTCG
CGACGCGCCATGGCGCGATCTTTGAGCAACCGGCGCCCCGTCCCTTCGCCGCAGAGG
ACCAGCAAAGACCGTCTCCCCAAGCGAGGGAAGAGGGAATTTTCGAAGAAGATGGG
CCCGAGCCACCTTACCTCTTCCTCCTCTTCCTCCTCTTCCTCATTCTCGCTGTCAGGGA
GGAGGGGACGTCTCGCCCGAAGGCTGGAAAGCGCGGGAAGTGAAAAATCCGCCGA
AAGCCCCGACCGGGACTCTGCCCAATTAGTGCTACCTGGAGAATGTCCCGATGTCGC
GCTAGAGCCACTCTGGCGAGTTTTGAGGGAGTGGGTCGAAACGGACGGTACAGACG
GGTCGGTTCAGTCAGTAACCGCGCCTACGGAGCTCCCAGACCTAGGCCGCGGCATTA
CCCTGAATGTACTCTCACGAGCGTTGGCCGTCGACCTCCATAGTTCCGAGGCTCGCGT
TTTTGTTCCAGAAGCTATTATCCCGTCCCCACCCAGTAGAGGACAAGTATATCATGGA
AGTGGCTATAGACAGTCTTTGGTCGATGACCCGCAGGTTCGGGAGGCCGCGGCCGAA
TTCCATGCCAATCCGAAATCAGCTTCCGTGTATCTCGAAGAGTACGGGATGCCAGTGG
AACAGTTAGAGCGCCTTGTGGAGGAGTTCATGGTCCAGCCCCTGTCGCGCGTTTCGT
ATGGGTATGGCGGCCCCAAGCTGAGCCCACGTGACAAACGGCTCTGCGAACTGTGCG
CTCTCCGCGGTCCGGGTTCTTCCGAGGGAGGATTGTCTCGTTCTGCGGTGCCGACGT
GCCTCCCGCACGTAGCGCACGCCATGCGTGATGGCCGTGTGTCATTAGCTCTGCCGCA
CCTTACCGAGCTCGTACGCGTGTGCCGCCGCTATGACCATCCGCAGAAAACTTACCTG
TTGGCGGCTCTTAGGAGAGCGTTTGTCCCGTTTGTATTTCCAGGAGCGGCCGTGGATC
CTACGGCGGCGTCTGCTTCTGGGGACCACCCTGTACTCGTTGGCGCCAGGCGAATAG
CGCGTGCCACGAGGGAGCTGGAAGCCGCAGCGAAAGAGCCACCTGCGGCAACCCC
GGAAAAACTTTCCTCTTCGGCGCTATTGCGCCATACTAGAACTTGCATTGTTGCTGCC
GCAGATCTCATGGCGGCGCTTACTGCTATAGACTGCCCACCAGTTACAATAAACCCCG
CCAAGCGTCGCCCGGATCTGTATCAAGCACTCTCGAGGGCGCCCAAAACAGTCTTTA
CGTGGGAGCGCGCCATGCGCTACTGCGCGGCTTCGCTATGGAGTCGCCTCGTCATAG
GGTCCCTGCCGCTGCCATCTACCGATGATGTCACTCTTCTTGTAGAAGCCATGGAAGC
TGCCGTGGGCGTGCTTGGCTGCTCCGACGAGGGTGACGTGCGGCCTTCAATTGCCTA
CGCGGCCTCTGGTTGGAGGATGATCGCTACCATGCTAGGAGATGATTCCGCGCCCGGC
AGCGAGCACGTCACGGACGATTGTCCCGCAGATGATGACACCCACCACCATGACAGG
CGCAAAAGACGAGGCGGCCGGCGGAGCACTTGCCGGTACGACCTACCTCCTGGCAC
TCCCGATCGCGCGGCCCACCTCCTACTCTACGGGAGTCCTGGCGGCGGCAACGGAGC
AGAGCCTTCGGCTGCCCAAGAATCTCCCGCCAACCCCTGGCCCAGAGCTCCACCGTG
CGATGAACAGGAGCCATTATCTGTGTCACCGTACGGGCCCGAACCCGACCGCCCTCC
AGATGATGACTTTGAGACCCGTAAAGGTCTGAAAAGAAAGTCATCTGAAGATCACGC
GGACCCGATCCCAGAAGGCAACGCGACTAAAAAAACATGTGGCCTCCAGGGCTTAC
CAGATTCATTACCTCCAGCGGTTCCAGAAACAGATAGAGACAACCTCCTCCTTTCGCC
ATGCCCCATAACTCCCGAGGGTCCACCATGTCCGCCCCGAGAAGAACCGCAAGAACC
ACAATCCCCGTCCTTTCACATTTCCGAGATAGGTGAGGCCCTGTTTCATTCTACCCCA
ATCAGCCCTACCATCCTGTTCGCACCGGAGGGTTTCATCCCGAACCAAGGGGATGTTT
ACCGTCCCCATGGCAAAAACTTGGGCGTTGCGCTCTCCGCAGGGGAGCCATACGTGC
CCGCCCATCGCGCGCGTCCTGCTTTGGAGCGGCTCGCGAACTATTTGCGCGGGTTGA
GCAGCGGTCCGGCGGGAAGCTATTCCGCGACCGGACCCGCAGATTGGCTACCTGCTT
TTGCGAGGGACCCAGTAGCCCTGGGGGAGTTTTGCAAGAGGATTTCACCCGTGAACA
GAGGGGGGCGCTGCGTCTTAGAGGAGCGGCTTTCCTGGACGTTGCGACATCCGGCTG
ACAAAATCTATGACCTCATCATTCTTGTCTCAGAACGTGCCCCGGGCGAATTTTTAGA
ACGCGCGTACGCCTACGCGGCCCTCTGCGGACGTGCCTGCATGCCGGGCGGTGCCTG
TTGGCCAGAATCGTGGACACGCGGGGAGTATCCCTATATCCCTCCCGACGAGAAAGA
CCCGGGCCGCAGCGTGGCCATTCTCTGTACGACAGACCTTGGATACGCCGGGGCGGT
AGAATGGATGCTTGAGCGCGCTCTCAGTACATCTGGGCGCGCATGCGTGGTCGTAGAT
GCTAGGGATCCCGGGGACGAGGATTCCCGCTCGGGTCCGCGCATACCTACCGGGCGG
AGGGGTGTTGTCTATGCCCGGACTAGTCCCCCGTCGCACGCTTACCTTGTAAAACTGT
TTACCGGTCCGGTGGCCGAACGGTTGGAACGGGGTGGGCTCAATGTAGGTCACGTAG
TAATGGACAGGCGACTCACTGTCTCATGGCCCCTCAGCGTTGGGAAGACCGACAACG
ACGATGAGGGTACAGGAGACAGAGAGAAGTGTCCTCCCGTTAAGCACCCCTTCCTCA
CTCACGTAACTCTCAGGAGACTACGCGACGTTGCTCGTGCCATGGATGCCGGGTGGA
GCCACTCCGAAATCGGAAAAGCTTCAGCCGTCTTGTATCCGTACATGGCCCAGCCCC
ATGTGGGCGCCGGGGCGATGCCGCCATGTCCGGACCTTTCCGAGTCCAGTTCCACTAT
GCATTCTTCTTCGTCGTCTTCCTCTTCCTCATGCTCCTCGTCGTCGTCTTCTTCAGACT
CCAGCTCATCTGAAGAAGACGGGGACGAGAAGAACGAGAAAGAAGATCGTGAGCG
CGCCGGAGGTGGAAAGCGGCGCGGCCGCCAGCGCCTCCTGATTCGGGACCGCGTGT
ACCGTGGCCGGGACTACCGCGTGCGCACTCCCTGCGCTGATATTGTGGGAGGCACTT
CCGCTTCCTTTAGGGCCGTCGCTGCGTGGGGGAGGGACACGGTAATTCCCGCCCTCC
CTCTCGCAGCATCCTGGTCCGATCCCAGCAAAATTCCACAGGAAGTTCTCCGCATCAT
CTCCGATTATTACCCGGATGCGCCAGGGGCGCGGGCGCGTCGGAACCCGCTTCACGT
GGAAGGACTAGCGCTTATGCGTGCCAGAAATCCCGCCCCTCTCGCATTGCTACTTGGT
GACGACTATTCCCATTATCACACTCCCCGCAATCGGTCCCCAATGTGTTGTTGCTGGA
GTGCTCCGCGGGGAGCAACGCCAGAGTCACCCAGATGCACCGGACACACGCGCACC
AGGCGCCCAAGGCTATCCGTCTCCTCAGGGAGGGGGGAAACTAGAACCCTTGTACCC
AGATTAGACTCTTCAGAGGATAGGCGGTCGCGTCAACGCCATAGACGCATGACTAAC
TACCCTCTACCGCATCCCACTGTCACTGACCCTTGGgTGGTAA.
SEQ ID NO. 6: gC (1245 bp)
TTATGTTGTCTTCCAGCACCATGCGCCCAAGCAAGCAACTAGTAACCCACCCACAAA
AGCGATACTAATTATAATAAAAATCGCAGCGACTGCGGGGAATCCTTGCCGCATTGGC
AGATTATCTATCATTAAGGAGGCGTTGAGATATCTGTATGCATGTTCATAGCCTCTTATG
ACGCACACGTAATTAGAGGCAGATTGAAATGTATCCTCCGGTGGTAGTTTTATGACGT
CCATTAGTGTAACGATATTTTTCCCCTCGGGCTCATCCAAAACAAAGGTCTGGTGTGG
GCTCGTCGAAATTCTATTATCTACGATCCATTCAATAAATCCTCGGCCGTCTATAATATT
AGCTGCTCTACACAGGGCATAGGCAGGAGGGACGAATTGTACCGTGATGCGGGGCTT
GCTAATCCAGCTGGGAGTCAACCTTAACAAGCGAGTTTCAGGTCGTAGCATTTCAAA
GTCCTCAGGAGGGGTCCATCGATATACAAATTGAATTTTGGAAGGGAGGGGCTGGCT
GGCTTCTCCACGTATTTCCAGTTCCCATGTCTGATCCACCGATCCATTCTCTCCTGATG
CATAAAGAACTCTTCTTTCTAAACTGTATTTTTCAGCAAGAGATGCATTATGTATCATC
CATGTACCCAGCGCAGCAGATGGAGGATAAGCGCCAAGCGCTTCAACACTTAAGTGC
GAAGCCTTATCTGAGTCTTCGTACAGTTCTTCTGATTTGATGCGGACAGTTGTAGGTG
GTCTTGCCAAAAAGACGTCGATAAAAGAGCTATTTAGTTTTGTAGACCCTCGACTTGC
GGTCCACTGATAACGCGCCATGTGAGATCTCATGCTAGACTTGATAGAGACATTGAAA
AAACCTGAACGACAGTCGTCCAAATATGGTGGGTTAATTCCATCTTCACCAATGTATC
CTAGGTCCTTGATTGAATCAGTAGCGGCATCATATGTGTAGAGAGTTCTTGGAGTAGC
TCTCCATGGATCTAGCGAAGGGGGGATCCCGTAGGCGCTTAGCGCAGTTTCAACTAA
GCTGGCTGGGTGTCGAATTACTTCTACTTTTACAGATTCTTCGTGGGTTAGAGGTCTGT
CGCTGAAGCATTGCAATAGAATGGGGTCACCGAGCTGCCCTCGAAAAACGTGTGGCG
GATCACAGAAAAATTCAAACGCAAGGCTTTGCAGGCTCAAGAGCAAAAGTATACTCG
AAACTAGCGCAGTACTCTGATGCTGCAT.
SEQ ID NO. 7: gL (708 bp)
TTATGGGCTGCTCGCATAGTCCGGGTGATAGTGGTTGATGTTCGTTTCCGGCCATCTGA
AATTTATGGTGGTCCTTTTATACAACGAATCAACAAGTCCACGAGCCAAGACGTTAGC
AGCCGCAAGCCCGGTAGCTTCCAGCCGGTCTTGAAGAAGTCTAATCCCCAAGTACGG
ATTCACTTTTGCAGCGTACCCATTCTTATAGTAGAAAACAACTTCCGTGTTTACGCACA
GAGGTCTGTAAATAATCACAGTCAAATTTTCAAAGTAGTCTCCCGATGGCAACGGCCC
GAAAATTCGCGCACCGTTATCTCGCGAATTACTATCACATGGTGCGGCCGCGGCAGAA
ACAGGATCACGAAAAATGCCAGGTATATCGACACTTGAACATAGCATAAGAGATGCTA
TTAGAAAAAAACAAGTCAACATTTTAAAAATCATTATCATATGGAGACCTAGGTCTCC
GTTCACGATCCGTGTTACGTCGACGAGCAGATGGAACTCTTTGCGCCCGACTTCTAAA
TGGATCGTCGTCAGAATCTGAGTCGTCCGAATACAAATGTAGTGGTCTACAGGAACC
AGTAGGTACTCTTTGTGGCGGACTTTTGAATGGATCGTCGTCCGAATCAGAGTCGTCC
CAATATAAATATGACGGCCTAGATGGAGTACTTCGGCTCTGTAGTCCGGAAGGACCTT
CACCTGCGTCAT.
SEQ ID NO. 8: gN (354 bp)
ATGAGGCTGCCGTGGGAAAGGAGTTTTATGCTGTTGGGAGCAGTGGTGTATTTGCTG
CATTTGGCACTAGTATTCGCGCAACCGGGTGTGACACCAGGACACGGAATGGAGCAT
GGGTATTATGGTCTTGGCGCTGGAAATATGCTTGGAGCGGAAAATTCGATGTATAGCG
CGAACATGGAACACTTTTACCACAGTGCCTGCTCGTCGCGCGGATTTTCCTTAGTAAA
CGGAACAGCTGCTTCCGTTTTCTTTTTCATTAGCCTGGCAGTGGCGTTGATTGGCCTG
CTGGCCGTGCTATATAACGGTTGTTTCAACAGCTTTAAAAGCAGTGTCATTAGTTCTC
GATGGTAG.
SEQ ID NO. 9: TK (1090 bp)
ACGAACCCATATCAGCATTGTAGCGCTCTACTGCGTCAATTAAGCTCGCGATCGCCGC
TCGTTTGGTTGTAAGAGAACTTGCCGTCTCGGCAAACTCTAAACATTTCAGTGCACAT
GCCTCCGGGGTGAGGCCATGTGCTGGTAAGTAAAAAATGCACAAGTTTTTCAATACC
GTCGCGAGTTTTTGAACCACAAATGAAAGTACAGTTTTTAATCTTTCCCCGTGAACAT
TTTGCTGATCAAAAGCTAATATTGCAAATAGCGTCTGGTCGATTGAAGGATTGATTTCT
GGAACAGTAGCAGTTTCTAAGCACTCTCTCTTGAATTTTAAGAGCGTGGCACTTGTTA
AGGCTTCTTGCTCCTTCAACAATGCTGGATAAGATGTACGTTGGAGGTAGGTGGTAGT
ATTCATGAGGCAGATGTACGAGTTACGTAATGCACACAAATACGTCCAGTCGACCGTT
TCCCCTGGGCGCCCCCTTTGTATTATACGCCGTGCCTGTTCGCAAATATCCAAATCCAT
TAAAACAATGTTGGAGTGTGTTGGAATGTCAGGAATTCCTGACAATAAAGTTATTAAA
TCTCCAAATTCGAGCGCTCCGCTTAGATATTGTGCAACAGGGAAACACAAACATGCC
GCGAGTGGGTGCCTATCTACGAGGAATAAGACATCATCTGGTCCGGATGCAGCTGCG
CTGTAGCAGCGTTCAGATCCCAAATAATTATAAATGACGTGGTTCATTATTCTTAGTGG
GGCAGCAATAGTCATCTGAACTTCCGCTACGCGACGAGACGCCTCCCGACATTCAAG
TTTTCCGGAAGCCTTTAGGGTTTGTATTTCATTAATTTCTTTGATCATATCCGCTCCGCC
AAACCATTGGCGCCATGCTTGCATAGGTTCCGCTAAGTATAGACGGCAACCTCTCCAA
TTTGGCATTTGCATCAACGTCTTTGCAGTGACTGTTTTACCGACTCCGAATGGCCCGT
CGACGTAAAGAAGTACAACGCGGCGAAAGTCGGAGTCATCGTTCTCGAACTCGGAG
CAAAACTGCACACCACCGGAAGTTTTCACGGCGCCAGCTACGGCCAT.
SEQ ID NO. 10: gM (1182 bp)
ATGAGCGGCTACTACTATAGTGGACAGGAATCGCGTTTAGAGAGGATTGCATGGCGCA
TGTGGCTGATTGAAGTAATCTCTTTTGTGGTTCTGGTGTTGATATCACTGGCCTCAGCT
TTCTCCACCCTTAGTCCGCAAGCAGGGTTTCCCTGTTTTTTCGGCAACGTCGCCGGTG
GACACCTTAAGCCAGCAACGCCTTTGATCAATCAACCTGGACTGCTAAGGGCGAACG
AAGATGGACTTTTTTTTATTTCGTCGCCAGGAATGATATTTACCGTTTACGCGACTATG
GCGGTGTGGATACTCTTGGCGATATACGTTACTCTTGGCGCAGTTAGAGCCAAGACTT
GTAATCACGATCGTTCATACGGAGCGTCCGAATTGACTTCGGCCGTGATCAGCACACC
TGCCTTAATGTCTATTATTATCACAGTATGGGCGTGGCAGTTGTTTGTACTACTCCTTTC
GTACAGACAGCTTACGCTATCAGCTGTCACATTTTGCATTATTTTTGTATCCGGCATCG
TATTTTTGGGTACGTTCGCGTCTTCCGGAAAGACACCTGACCAGTACTCAGTTTTTAA
CTCACAGCTCAAAAGTGCATGCCCTGAAGTTCATTGTGTTGTATCACCGTTTAAGGCA
GTGGTATTAAACGTCTACGCCTCTATCATGGGAGTTTGGGGGCTTATAATGGCCTTGTC
CGGAGCACTCGTCATGACCGTGAATTTTGGAGTCAGTGTCCCTGCTTCTACACTTGGG
GCAATGATTGCATTTCTTATCATAGGTATCATATACTTGATTGTGACCGAGCTGGTTTTA
GCGAAGTATGTGCATGTACTTATTGGTCCGCATTTCGGACTCATCATTGCTCTTGGAAT
GCTTGGGACGGCATCCCTGCACTATGCTAATACGTTGGATAGCGGAACTCATCCCAAC
TGGAAGCCGGCTGCGGCAGGAATTCTAGGAGTATTTGCCCTCCTTGTTTTAGTTTTGG
CTATTCTGCGAGTCGTACGGGCATACAGATATCACCAGTACAGACAGACGGATTTCAT
CCGCAAGGTAACGACCGTTGCCACAGAAGCTTTAGATAAAACCGTGAAACGCAAAA
CGCGCCGCGAAAAATACAAAAGCCGAAGAGACAAGCACGTTTATGAAACTGTACCC
TATGACGACGAACTGTAA.
SEQ ID NO. 11: gD (1305 bp)
ATGCACCGTCCTCATCTCAGACGGCACTCGCGTTACTACGCGAAAGGAGAGGTGCTT
AACAAACACATGGATTGCGGTGGAAAACGGTGCTGCTCAGGCGCAGCTGTATTCACT
CTTTTCTGGACTTGTGTCAGGATTATGCGGGAGCATATCTGCTTTGTACGCAACGCTAT
GGACCGCCATTTATTTTTGAGGAATGCTTTTTGGACTATCGTACTGCTTTCTTCCTTCG
CTAGCCAGAGCACCGCCGCCGTCACGTACGACTACATTTTAGGCCGTCGCGCGCTCG
ACGCGCTAACCATACCGGCGGTTGGCCCGTATAACAGATACCTCACTAGGGTATCAAG
AGGCTGCGACGTTGTCGAGCTCAACCCGATTTCTAACGTGGACGACATGATATCGGC
GGCCAAAGAAAAAGAGAAGGGGGGCCCTTTCGAGGCCTCCGTCGTCTGGTTCTACG
TGATTAAGGGCGACGACGGCGAGGACAAGTACTGTCCAATCTATAGAAAAGAGTACA
GGGAATGTGGCGACGTACAACTGCTATCTGAATGCGCCGTTCAATCTGCACAGATGTG
GGCAGTGGACTATGTTCCTAGCACCCTTGTATCGCGAAATGGCGCGGGACTGACTATA
TTCTCCCCCACTGCTGCGCTCTCTGGCCAATACTTGCTGACCCTGAAAATCGGGAGAT
TTGCGCAAACAGCTCTCGTAACTCTAGAAGTTAACGATCGCTGTTTAAAGATCGGGTC
GCAGCTTAACTTTTTACCGTCGAAATGCTGGACAACAGAACAGTATCAGACTGGATTT
CAAGGCGAACACCTTTATCCGATCGCAGACACCAATACACGACACGCGGACGACGTA
TATCGGGGATACGAAGATATTCTGCAGCGCTGGAATAATTTGCTGAGGAAAAAGAATC
CTAGCGCGCCAGACCCTCGTCCAGATAGCGTCCCGCAAGAAATTCCCGCTGTAACCA
AGAAAGCGGAAGGGCGCACCCCGGACGCAGAAAGCAGCGAAAAGAAGGCCCCTCC
AGAAGACTCGGAGGACGACATGCAGGCAGAGGCTTCTGGAGAAAATCCTGCCGCCC
TCCCCGAAGACGACGAAGTCCCCGAGGACACCGAGCACGATGATCCAAACTCGGAT
CCTGACTATTACAATGACATGCCCGCCGTGATCCCGGTGGAGGAGACTACTAAAAGTT
CTAATGCCGTCTCCATGCCCATATTCGCGGCGTTCGTAGCCTGCGCGGTCGCGCTCGT
GGGGCTACTGGTTTGGAGCATCGTAAAATGCGCGCGTAGCTAA.
SEQ ID NO. 12: gB (2620 bp)
TTATTCGTCTTCGCTTTCTTCTGCCCACGAAGGGTATTCGAGCAGGGGGTAGTCATCTT
CCTTGGTTCGGGGTTTTCTGCGAAATAGGTTTCTCATATTTTTAGCAAAAGCAGAAAA
CCGCGCTTTCTGTCTTCGAGCTTTCTGTTCATCCTTGTGTAGAAGCTCAAGGCCTTTC
AGAACTCTGGTGGCAAGTATCCTGTCGTCCTCATCACTATCCTCCTCAACCTCCTCCTC
ATCCTTGTAGTAACGTCTAGAGGGTCGTGGAGGAGTTCCGGCCGGGGGAACTGCGCC
TGGGAACAGAACCTGAACTGGGTTTGATTTCAGGTTCATTACATATTTGAACGCCAGC
AGTCCTAAAATAATGCTCACCACCACCGCTATCCCAATTCCGAGTGCGGCGAACGGGT
TAGAAAGAAATGAGGCGATGCCAGATACTGTAGAAATTACTGCTGCCGCGGCTGTCAT
CACTACAGTTCCCAATGCCTTTCCTACTTCTCCAAGAGTGTTGCCAAAAAAATCTGCT
ATTGCTCTAAAGATTGCATCTCCCCTATCTCCACGTATTACAGTGTCAATGTCTCTGAA
CCTTTTGTTATAAATATTTTGATATCTGACCACATCATCATAGTTTAAAGTCCCAGTATC
TCTGAGTTCCTCGCGAGTGTATACTTCGACGGGCACAAAGTCCAAATCTTCTAGTATA
GTGGCGTTCAAGTTGATGAATATGCTCACTTCTTCGATCTCGGAAGCATTTACTTGTCT
AACAAATGTATAGTCTTCGTAAAGAAGGTAGTTTTCTCCCAACAGAAAGTACCGTCTG
TGATTGATCATGCATGGTTCTATCAAATTCCGCCCTTGTAAAATTTCATTATGTTCTCCG
AGTTGGCCTAATATGTCAAGATTGTGGTTTTCTGTTGAGTTCGCAGAAAACTGTGACT
CAGGGGAGGACGAATACCTGAAAATAAGTACTGGTCTGGTATAGCACATGGTTGGGT
CCCCTGGCATGCGCATGGAATCCTGCATCCTAATATTTTCGATTGGAATTTCTATACATT
TTGATACCGCTACGATGTCTCCCAATAGCCTGGCGCTTACAGGTTGTCCGAACAAAGA
TGTCATCAGTGAGTTCGGGTTTAGTTTCTTCATCTCATGCCATACAATCAGTTGGCGGT
TCTGAAGCTCACACCACGCTTCCAACAAATTTCCGATAAGCTCGTTAACATGGGCTTG
GATTTTGTCGTATGCAAATTGTAACATGGCAAATGTTGCAGAACTGTGGGTAGTAATT
CTATCACCCTGAGGTCCAGACTGCAGCGACGCCGTGCGGCGACCTGCGGCTTGGCGA
CGCTCTCTCCCTCTCGGGAGATGATTTTGTCTTTGTGCCTCTTCTAGGTACATTTCAGC
CAAGCCATGGCTCATGAGTTTCTGAAATGCGATCAGAAATCCGCCACTACCGAGATAG
TATTCGATGTCCCCAGAACGGACATGAGTCGAACTATACTTTCTGGCAAAGATGCCAT
CTATGGCCTCCGAGGCTATGGTAGGAACGCATTCAGCCAAATGAAGCCTGCTGATGTT
AAAAGGTTGTTTTCCGGACGAGAACGTCATAGTCATATCTTTAAGTGAAAAGTGGTAA
CTGTTTTTGTACGAAACACGAACTGCTTCCGGGACTTCAATCCATTTACTGAGAGTAC
ATACAGATTCCTTTTCTTCCATTGCATCCCAGCCTATAGTGAATTGTTCATCTGTTAGAA
AGTTTCTTTTTTTAGGGGGTCTTATTTGTCCGGTTTCCAAATCCCTGACTTGATAATTT
GCGATTTCGAGAAATCTATAGTCTCTGTAGACACTGTGACGCCTTGGTCCGGTCGTGT
TTTTGGTATAAAAAGGAGAAATTTCTACTGTATCACCTGTCGCCATTCCAAAGTAATCA
TACGGGTATACAGATCTAGCCTGTAGATATTCCACAACACAGTCGACCGATGTAGACG
TTCGGTATCCCAGGGTTTGATGTCGCTTAGTAAAGTTAGTTGTATGAAACGCCTTGGA
GACAGTTGATCTTAACAGTGATGGAACCAGGGGCAATTTTTTTTCTGCTTCATCATTGT
CGTAAGCTTCAAAAAACATGAAATTTTTATGATACGTTGCTTTGGATGAGCATTCTCCC
GATCGATCAATCCTGACAATTTCATGATAGTCTATTGGAACCCTGGTCACGTACTCATT
TGTTATTTGGGGTCTTGAGAATAATGCCCACGTAGTAACTGTGGTTATATGTTTATAGTA
TAGAGTCACATTAAAGACGTACGGGGCAATGTTTTGCTTGAAGACTACGGCAATTCCT
TCAGTCATGTTAGTCGAATCGGCATGTCGGTGACAATGTCGTGGCTGGGCAAGCCTC
GCAACACTGGCGCCAGTTGGAGATGAGCACGCAAAAATTCTGTTTCTAATCTGGGGT
TCTTTCGGTGCCCCCGACCCAATGGCTTCGGAGAAAGAGATTTTTCCAACATCCATGC
TGGCTGTATCACGAGGGTCTATTATTCCAGCAATTCCGTGTGAATCTTGAGATAGGGTA
GATGGGATCAGGATTGCCACGCACACGCAGATCAGCATTTTCAAGCTAGCC.
SEQ ID NO. 13: gH (2415 bp)
TTAAAGTTCGCTCTCCTCGTCAGACGGTAGCCGTACACGTCTAAATTGCGTTGCCGAG
CTGAGGCAGAATTTATATATAGCCCTGATGGCACTCCATAGAGCAACCAATCCAACAA
GTGCCCCTACCACAATACCGATAATGTATGCCTTGTCAAAAACTATTATGCTTCTGTCA
AGTCCTAAAATTCGGACAGCAGTTCCGTGTGGTCCTAGAAGCGCGTAGTTATAGAACT
CGGGGTTCATAGACTTAAAAAATGCAATAGATGAGTTGGCACCGGCTAACAGTTCTG
ATTGAACATATGCACTGTCTATAAAGAAAGCATCCTGGAACCCCCGTAGACCGTATTG
AACTATCAGGTGGTTGCAATATATGCAAGTTTTCGCTCGTCCATTCAAATGACCATCCT
CCATTTTTGCTGTTCGCGCGATTTGCGGTAGGGAGTTGCAACTTGAATTCACAAACGT
TACAAACAAAGGGGCAGCTGTTGAGGTATCCGGAAGTCTGTAGGTCATTCCAGTGCT
CGGCGGGCTACTTGTCATCACCCAACTAGAATGCGCGGTAACAGGGAGGAAAAATAC
GATGTGGTGTTCCAGGTCCCAGTTTAAACATTTCTTTACCTCTGGAAAAAACGCAGTT
GCCAGGATCTGAGGGGTCAACAGAGCTTGTGGCGGTATCGGTGCACTATATGATGGC
AATTCACCTTCAGATGATAAATATATTTTGGTGTAAAAAAATCGAAATGGAGCAGATG
CTGTCAGAATTATTTTCCATGGTGCACTTCTGGAAATATCAAACCTTGTCGCAATTGCA
CAAGGAGAAAACCACTCTTCAATTTTCGATGCCTGTCCTTTCTTGAAGCTCCCGTCTG
GTTGGGAGTGAATCTCGTTGATATCGGCTAGCCGGTCGACAGAGCATAAAATTGTCAT
GCGCCTGAGGAAGTTCCTTCCATGTTGTATAGAAAGTTGAGGAGCCCGCAAAATAAT
GTACGTAGAAACTGCCAATAACTTTCTGTACGCGGGGGTCATTACCTTTTGTGTAATG
ATTTCTCTGTAACTGTTATCTATTATTTCAAGAGCCAGTTGGCTGGGGGTCACTCCATC
GATGCTCGCCAGGAATGCATTTTGGGGATCTTGTTCTGCAGTGATAAGCACCGCATTC
ATGATAGTAAGAAGCAGGTTATGATTTTTTATCATGGGTTCCCATGGCCTGTCGTGCAT
AATTTTAAGAATACTGGATGGGATAGAAGTAAGAGCACCACCGACATTCTGAATAAAG
CGAATATTCGTTGTATAGTTTACTGAATTCGCCCCCCCAAAACACACTGCGACTTGGT
CTGCCCATGTTTGCGTTTCTGCAATAAACTCGACGATCTCCGAATCAGTTACGTATGCA
TCGATCGTAAACTTTAGAAATCGAAACCACGCGGCCATAAACCGTGCTGCGAATCTTA
CAAAAAAAGACTCGTCTATGGTATTCGGGCAACCTGCGATTGCGACCTTCTGCAAAG
TTAAAAAGTGTCCCGCGGATATTTGTTCGTCGAAGCTGGCCAGCTCTAAGAAATCTTG
AGCAGCGGTCGTTACTGCTGTAAACAAATTTGGCCGATAATTTGGTCCAGCTGCGACG
TTGATGCGCACAGTTAACCCCTGATCTTCAGCCTTCAGCATATAGGGCCATGACTTCA
GGGACGTTTCTTTAACCTTCGCGCTGGTTGGTGTAATTATCATCTCTATTTGAGGAGAA
TGCTGGGTCGCGATTATAAGGGTCGCCACATTCAGTCCGATGTTAATCTTAAGATCTAT
GTCACTAGTAACTGCGAGGCTAATATATCGTGTAAACACAAATGGAAAAACAGTTTGT
AAAGGATCACTGTAAATTACACGAATCGGAGCGATGATGCTCTCGCCCCTATGAACTG
AGTTATTTGGTGGAAAGTTGACGAGTTTCGCAGATGTAAGTGCATTCCCCTTGTATCC
TACCAAAAGTCGATGCTCAAGCTTTGGATCGTTGCTGAATTTTCGTGGTTGGATGGTA
GCCTCGCTAGCAGAACCCCATCTCGAGTAAGTATAGGCTACGTACAGTTGTCCAAGCA
AATCTAGAGCGATCGATAAATTCCTGTTAGCGCTTCCAGGAGGCCCAAAGATTAAGTT
TCGATCGACAAGGCATAGTTGTGTGATGTGGCCGTTTTCGACTGCTGTCGCTATCGCA
ATGAATTCTTTCGGAGGGGCCAAATTTTCACTGGTGGGGGGCACGAAGACAATTTTT
GCCTTTGCCTTGTCATCCCCTTTAGCTCTAAGTAATATCGCCAGAATGAGGAAAAATAT
TTCTCTCGCGTGTCCCATCGGCGCGGGTTGCTGGTGAAGCCACGCTCTCTCGAGTAA
GAATGAGTACAAAGCAAGGAAATGAGTAAAGGACAT.
SEQ ID NO. 14: gI (1089 bp)
TCACATTTTTATTGAGTCGGGCGAGCTTAGCGCAGACGGGTTAACAATCGCAACCAGT
TCCACAAGTTCACTATCCTGGTCAGCCCCCTTGTTTATTTCGCACGCAACCATGGGTC
CAAATTTTCGCGATTCCCTTCTAGTTTGGGAACGTTCTTCGTCGTCTTGCGAGACCGT
GTCTAATTTTCGGTGCGGGGTGCACATGTTTCTGGTGACGATGGAAATTACAATGACG
AGGCCGATCGTTGCGCTCACGACGGCAGCCACGGTTACCCCAATTAGCGTAGGGCTC
ATTGTCCCGAGACGGACAGTAACGTTTGAATTTTCGTTTGCGGGTGTCGGTTCGTAAT
GATCCACGCCATTTTCTAGCCAGGGAAAGGTAAAGTGTTCCGCTTCAAGTTCGGAGG
CGCTGGTTGCAGTGACGGGCGTCGGGGTTGTCGCCTCGGCGCTGGTCGTCGTGGCCT
CCGTGGCAACGTACGCTTGCCAGTTGCGCCAACTTTCCTCGGTCCTGAGATAGGCCTC
AAGTTGGACGGTAGGTAATCCGAAGGTGCGACACGAAGCCTTGGAATAGAGTCCGC
GAGTCGCGGCGGTGTTCGCGAATTGATACACCGATAGTTGGATCGCGAAGACGTCAA
TGGGTTCTTTGGTGTCGTCTAGCCGAACAATTACGTAGAACATCCCGCTGTCGTTCGG
ACGAGGATTTCGTATAGTGAGCTTAAACGGGTGTGGAGGTCCGGTTAGTCTGTTCGA
GGCGGAGAACGTGTACTCGGTCACTTTACAGCCGACAAAAACAGAAGACGATCGAT
GAGTGCAATTTTCAAAGCGCTCCTCGCTAAGTTCCTGGTAGCACTGGTTTGTTATATC
AGACCGAAACGCGACGCGGACGGTTCCTGAGTAGGGTTTGTGGGGTCTCTGGCCAG
GCATGAAAAATAGCTGCAGTTGAATTGTAGCTTCGGGGCGAGGCGCGACGATCACGA
TGTGATCGTCGTCAATCCTGGCGGAGACGTGATTTCCAGTGATCACGATTCCCATCGC
GCCGAAGGGTGCGAGCGTCGCGGCAAGGAGAGCCAGAGTTCCAAGTAGCGATGCCA
T.

### Example 7: Determination of Immune Duration of ILTV FJ19 Strain

Seventy 14-day-old SPF chickens were divided into 2 groups. The 30 chickens in one group were vaccinated with the virus of the FJ19 clone (P1-generation) via eye-drop vaccination at a dose of 10^{3.0} EID₅₀ per chicken. The 40 chickens in the other group were not vaccinated. At 4, 5 and 6 months after the inoculation, 10 chickens each taken from the two groups were subjected to intratracheal challenge with the virulent strain ILTV WF03 at a dose of 0.2 mL/chicken, with a virus content of 10^{4.0} EID₅₀, and clinical symptoms were observed for 14 consecutive days after the challenges.

The results showed that no visible clinical symptoms were observed in non-challenged chickens at the observation period. At the observation period after the challenge, the non-vaccinated chickens had the morbidity of 100% (10/10), 100% (10/10) and 90% ( 9/10) after 4, 5 and 6 months, respectively, showing typical clinical symptoms such as hemoptysis and mouth breathing. However, the vaccinated chickens had the morbidity of 0% (0/10), 0% (0/10) and 10% ( 1/10) at the observation period of the challenge after 4, 5 and 6 months of vaccination, respectively.

Although the examples described above have provided a detailed description of the present disclosure, they are only a part of, rather than all of the embodiments of the present disclosure. All other embodiments that can be obtained according to the examples of the present disclosure without creative efforts shall fall within the scope of protection of the present disclosure.

1. (canceled)

2. (canceled)

3. (canceled)

4. (canceled)

5. (canceled)

6. An infectious laryngotracheitis vaccine, comprising a live virus of a natural attenuated strain of infectious laryngotracheitis virus (ITLV) with good safety and good immunogenicity, designated as ILTV FJ19, having a deposit number of CCTCC NO: V2023109.

7. The infectious laryngotracheitis vaccine according to claim 6, wherein administration of the infectious laryngotracheitis vaccine comprises oral administration and eye-drop vaccination.

8. The infectious laryngotracheitis vaccine according to claim 6, wherein the live virus is obtained by inoculating an SPF chick embryo with the natural attenuated strain of ITLV via an allantoic cavity route, and, harvesting an chorioallantoic membrane and/or an embryonic body after 5 to 7 days of inoculation.

9. The infectious laryngotracheitis vaccine according to claim 7, wherein the infectious laryngotracheitis vaccine is used for vaccinating chickens, and an immune-protection rate for challenging with a virulent ITLV strain ILTV WF03 reaches 100%.

10. A method for preparing the infectious laryngotracheitis vaccine according to claim 6, comprising inoculating an SPF chick embryo with the natural attenuated strain of ITLV as a vaccine producing strain via an allantoic cavity route, harvesting an infected embryonic body and/or chorioallantoic membrane after 5 to 7 days of inoculation, and performing grinding to prepare a virus liquid, such that the infectious laryngotracheitis vaccine is obtained.

11. A method for preventing infectious laryngotracheitis, comprising administering the infectious laryngotracheitis vaccine according to claim 6.

12. The method according to claim 11, wherein the vaccine is a natural attenuated live vaccine.

13. The method according to claim 11, wherein the infectious laryngotracheitis is an avian infectious laryngotracheitis.
